(19) United States
(12) Patent Application Publication
(10) Pub. No.: US 2017/0158636 A1
(43) Pub. Date: Jun. 8, 2017

(54) FUNCTIONALISED AND SUBSTITUTED CARBAZOLES AS ANTI-CANCER AGENTS

(71) Applicant: Novogen Ltd., New South Wales (AU)

(72) Inventors: Ian James, Hornsby (AU); Ian Dixon, Hornsby (AU); Andrew Heaton, Hornsby (AU); Eleanor Eiffe, Hornsby (AU); Peter Gunning, Sydney (AU)

(21) Appl. No.: 15/323,682

(22) PCT Filed: Jul. 16, 2015
(86) PCT No.: PCT/AU2015/050399

§ 371 Date: Jan. 3, 2017

Related U.S. Application Data
(60) Provisional application No. 62/025,209, filed on Jul. 16, 2014.

(51) Int. Cl. C07D 209/88; C07D 405/12

(52) U.S. Cl. CPC C07D 209/88; C07D 405/12

(57) ABSTRACT

The present invention relates to anti-tropomyosin compounds, processes for their preparation, and methods for treating or preventing a disease or disorder, such as a proliferative disease (preferably cancer), using compounds of the invention.

### FIELD OF THE INVENTION

The present invention relates broadly to pharmaceutical agents as treatments for proliferative disease such as cancer and a range of degenerative diseases such as osteoarthritis, atherosclerosis, heart disease and inflammatory bowel disease. In particular, the present invention relates to pharmaceutical agents which comprise aryl and/or alkyl substituted carbazole compounds. The invention further relates to methods for treating or preventing a disease or disorder, such as a proliferative disorder (preferably cancer). The invention also relates to processes for preparing the compounds.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Cancer kills many thousands of people and is the second largest cause of death in the USA. There have been significant breakthroughs made in treating or preventing a wide variety of cancers. For example patients with breast cancer have benefited from early screening programs as well as a variety of surgical techniques. However, these often prove physically and emotionally debilitating. Moreover, patients who have undergone surgery and subsequent chemotherapy often experience a recurrence in their disease.

A potential new method of specifically attacking cancer cells is through disruption of cancer cells' cellular skeletal system comprised predominantly of actin. The actin cytoskeleton is intimately involved in cell division and cell migration. However, actin plays a ubiquitous role as the cytoskeleton of tumor cells and the actin filaments of the muscle sarcomere. The differing roles but similarity in structure make actin a hard target for drug development, due to unwanted off-target side effects.

### SUMMARY OF THE INVENTION

The invention seeks to address one or more of the above mentioned problems, and/or to provide improvements in therapy (e.g. cancer therapy) and in one embodiment provides an anti-tropomyosin compound.

In a first aspect of the invention there is provided a compound of general formula (I), or a pharmaceutically acceptable drug or prodrug thereof:

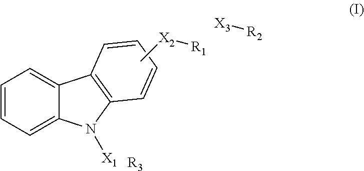

wherein:

R₁ is

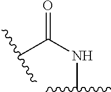 [SMILES: *NC(*)=O]

or a 5- or 6-membered carbocyclic ring wherein between 1 and 3 ring carbon atoms may optionally be replaced with S, N, O, NH or NR₅ and wherein the ring may optionally be substituted by R₆;

R₂ is a monocyclic or bicyclic carbocyclic ring having between 5 and 10 ring carbon atoms wherein 1 or 2 ring carbon atoms may optionally be replaced with S, O, N, NH or NR₅ and wherein the ring may optionally be substituted with R₆, or R₂ is

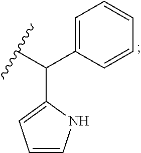 [SMILES: *C(c1ccccc1)c1ccc[nH]1]

R₃ is H, halo, NH₂, N(R₅)₂ or a 3- to 7-membered carbocyclic ring wherein between 1 and 3 ring carbon atoms may optionally be replaced by S, N, O, NH or NR₅ and wherein the ring may optionally be substituted by R₅ or R₆;

X₁ is absent or is an alkyl group having between 1 and 10 carbon atoms, or an alkenyl group having between 2 and 10 carbon atoms;

X₂ and X₃ are independently absent or selected from the group consisting of: S, O, NH, N(R₄), C(O), C(O)NH, an alkyl group having between 1 and 10 carbon atoms, an alkenyl group having between 2 and 10 carbon atoms, CH(R₄)CHC(R₄)C(O), (CH₂)₀₋₅C(R₄)C(R₄)(CH₂)₀₋₅, and a 5- or 6-membered carbocyclic ring wherein between 1 and 3 ring carbon atoms may optionally be replaced by S, N, O, NH or NR₅;

X₄ is O, NH, NR₅ or S;

R₄ is H or C₁-C₆ alkyl;

R₅ is CH₃, (CH₂)₁₋₅CH₃, (CH₂)₁₋₅OMe, CF₃, CN or OCF₃; and

R₆ is H, OH, alkyl (e.g. C₁-C₆ alkyl), C₂-C₆ alkenyl, halo, alkoxy, amino, alkylamino, dialkylamino or a dioxolane ring fused to 2 adjacent carbon atoms of R₁ or R₂.

X₁ may be an alkyl group having between 1 and 10 carbon atoms.

R₃ may be H, NH₂, N(R₅)₂, halo, or a 4-, 5-, 6- or 7-membered carbocyclic ring (e.g. cycloalkyl or aryl) wherein between 1 and 3 ring carbon atoms may optionally be replaced by S, N, O, NH or NR₅ and wherein the ring may optionally be substituted by R₅ or R₆.

X₂ and X₃ may be independently selected from the group consisting of: S, O, NH, N(R₄), C(O), C(O)NH, an alkyl group having between 1 and 10 carbon atoms (e.g. between 1 and 5 carbon atoms, such as CH₂, (CH₂)₂ or (CH₂)₃), CH(R₄)CHC(R₄)C(O), and a 5-membered carbocyclic ring (e.g. aryl) wherein between 1 and 3 ring carbon atoms (e.g. 1 or 2 ring carbon atoms) may optionally be replaced by S, N, O, NH or NR₅ (e.g. N and/or O)

R₁ may be

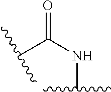 [SMILES: *NC(*)=O]

or a 5- or 6-membered aryl or cycloalkyl group wherein between 1 and 3 ring carbon atoms may optionally be replaced with S, N, O, NH or NR₅ and wherein the ring may optionally be substituted by R₆.

R₂ may be an aryl or cycloalkyl group having between 5 and 10 ring carbon atoms wherein 1 or 2 ring carbon atoms may optionally be replaced with S, O, N, NH or NR₅ and wherein the ring may optionally be substituted with R₆. R₂ may be:

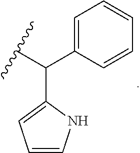 [SMILES: *C(c1ccccc1)c1ccc[nH]1]

In one embodiment, the compound of formula (I), or a pharmaceutically acceptable drug or prodrug thereof, is:

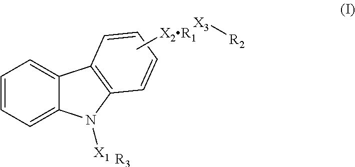

wherein:
R₁=

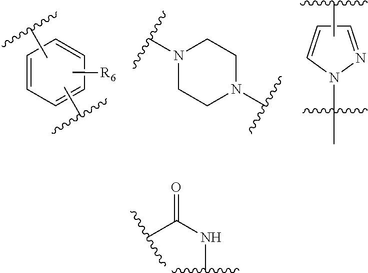

X₁=absent or (CH₂)₁₋₅
X₂ and X₃═O, NH, N(R₄), C(O), C(O)NH, (CH₂)₀₋₅, CH(R₄)CHC(R₄)C(O), pyrazole, isooxazole
R₂=

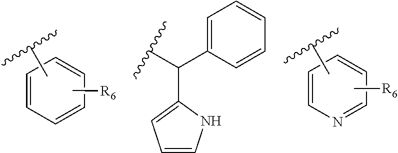

X₄═O, NH, NR₅
R₄═H, CH₃, CH₂CH₃

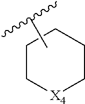

R₅═CH₃, (CH₂)₁₋₅CH₃, (CH₂)₁₋₅OMe, CF₃, CN, OCF₃
R₆═H, OH, alkyl, halo, alkoxy, amino, alkylamino, dialkyl amino, or a dioxolane ring fused to 2 adjacent carbon atoms of R₁ or R₂
R₃═H, NH₂, N(R₅)₂, halo

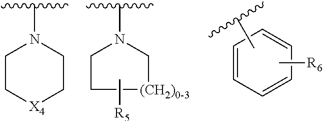

In one embodiment, the compound of formula (I) is a compound of formula (Ia):

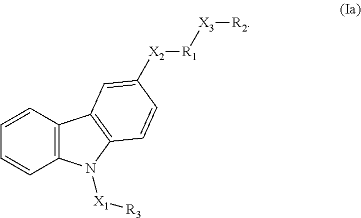

In another embodiment, the compound of formula (I) is a compound of formula (Ib):

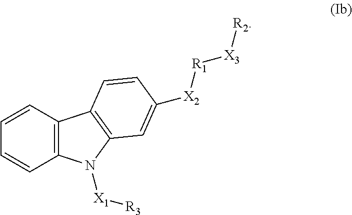

X₁ may be an alkyl group having between 1 and 5 carbon atoms (e.g. CH₂, (CH₂)₂ or (CH₂)₃).

R₃ may be N(R₅)₂. R₅ may be selected from CH₃ and (CH₂)₁₋₅CH₃ (for example, CH₂CH₃).

R₃ may be a 4-, 5-, 6- or 7-membered cycloalkyl group wherein between 1 and 3 ring carbon atoms may optionally be replaced by S, N, O, NH or NR₅ and wherein the ring may optionally be substituted by R₅ or R₆, such as:

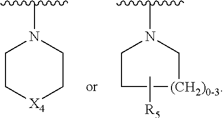

R₃ may be a 6-membered cycloalkyl group wherein between 1 and 3 ring carbon atoms may optionally be replaced by S, N, O, NH or NR₅ and wherein the ring may optionally be substituted by R₅ or R₆, such as:

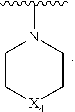

X₄ may be NH or NR₅. R₅ may be C₁-C₆ alkyl (e.g. CH₃ or CH₂CH₃)

X₁ may be absent, and R₃ may be H.

R₃ may be halo (e.g. chlorine).

R₃ may be a 5- or 6-membered aryl group wherein between 1 and 3 ring carbon atoms may optionally be replaced by S, N, O, NH or NR₅ and wherein the ring may optionally be substituted by R₅ or R₆, such as:

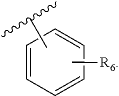

R₆ may be halo (e.g. fluorine).

X₂ may be C(O).

R₁ may be a 5- or 6-membered cycloalkyl group wherein between 1 and 3 ring carbon atoms may optionally be replaced with S, N, O, NH or NR₅ and wherein the ring may optionally be substituted by R₆. R₁ may be:

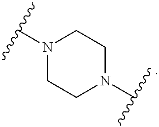 [SMILES: *N1CCN(*)CC1]

Accordingly, the compound of formula (I) may be a compound of formula (Ic):

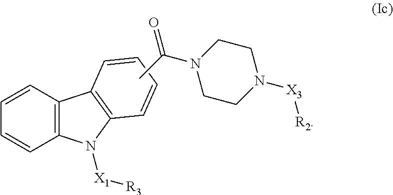

X₃ may be (CH₂)₀₋₅ (e.g. CH₂, (CH₂)₂ or (CH₂)₃).

R₂ may be an aryl group having 5 or 6 ring carbon atoms wherein 1 or 2 ring carbon atoms may optionally be replaced with S, O, N, NH or NR₅ and wherein the ring may optionally be substituted with R₆. R₂ may be:

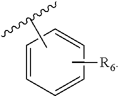

R₆ may be selected from H, halo, alkoxy and a dioxolane ring fused to 2 adjacent carbon atoms of R₂. R₆ may be halo (e.g. fluorine). R₆ may be alkoxy (e.g. methoxy or ethoxy).

The compounds of the present invention are exemplified in the following structures:

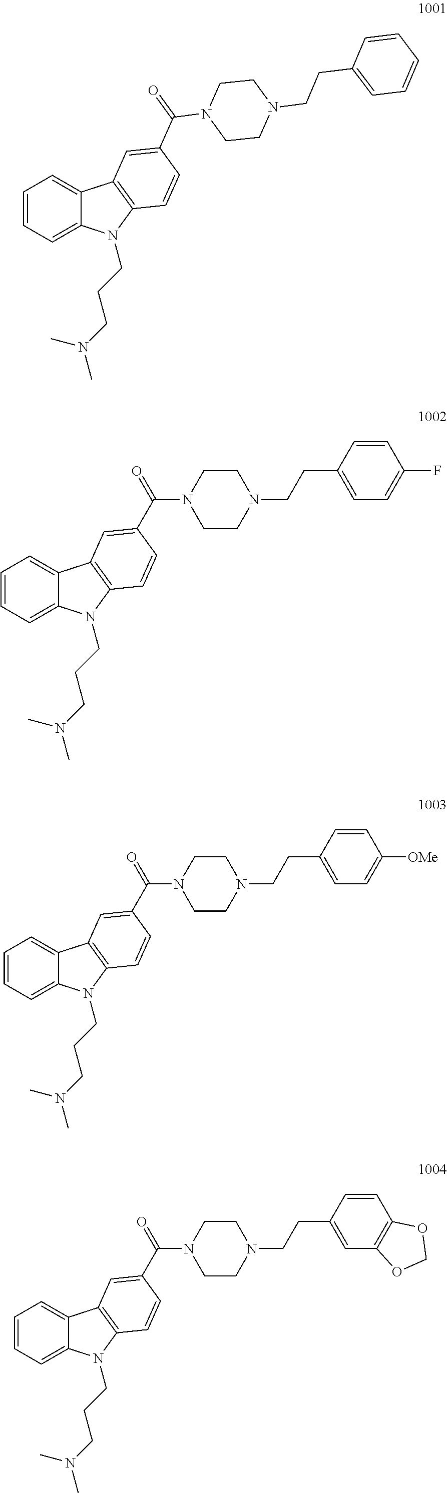 [SMILES: CN(C)CCCn1c2ccccc2c2cc(C(=O)N3CCN(CCc4ccccc4)CC3)ccc21 ; CN(C)CCCn1c2ccccc2c2cc(C(=O)N3CCN(CCc4ccc(F)cc4)CC3)ccc21 ; COc1ccc(CCN2CCN(C(=O)c3ccc4c(c3)c3ccccc3n4CCCN(C)C)CC2)cc1 ; CN(C)CCCn1c2ccccc2c2cc(C(=O)N3CCN(CCc4ccc5c(c4)OCO5)CC3)ccc21]

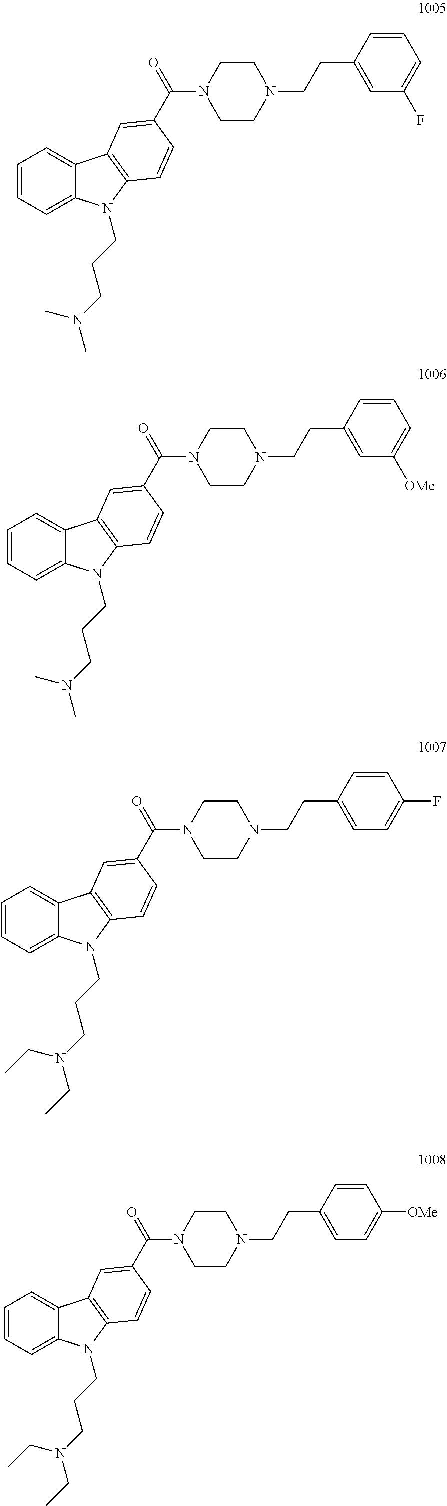 [SMILES: CN(C)CCCn1c2ccccc2c2cc(C(=O)N3CCN(CCc4cccc(F)c4)CC3)ccc21 ; COc1cccc(CCN2CCN(C(=O)c3ccc4c(c3)c3ccccc3n4CCCN(C)C)CC2)c1 ; CCN(CC)CCCn1c2ccccc2c2cc(C(=O)N3CCN(CCc4ccc(F)cc4)CC3)ccc21 ; CCN(CC)CCCn1c2ccccc2c2cc(C(=O)N3CCN(CCc4ccc(OC)cc4)CC3)ccc21]

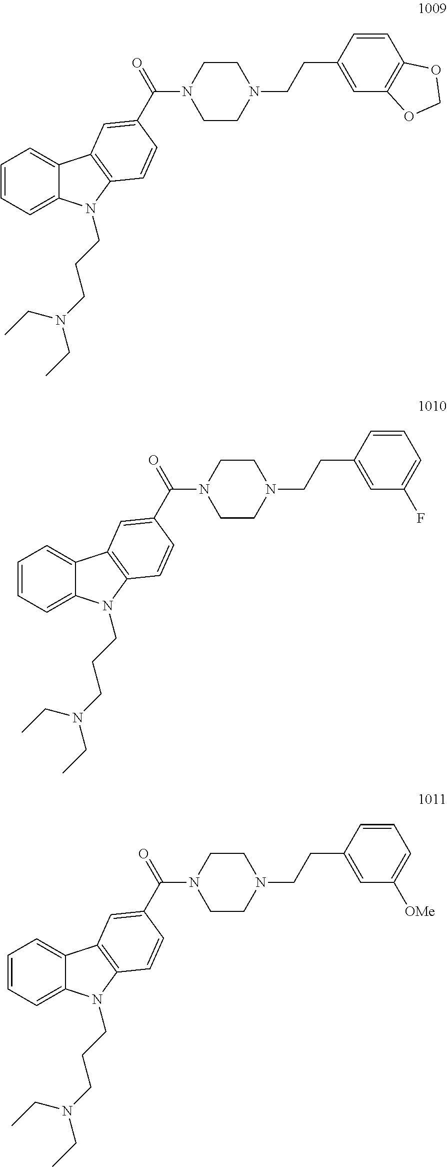 [SMILES: CCN(CC)CCCn1c2ccccc2c2cc(C(=O)N3CCN(CCc4ccc5c(c4)OCO5)CC3)ccc21 ; CCN(CC)CCCn1c2ccccc2c2cc(C(=O)N3CCN(CCc4cccc(F)c4)CC3)ccc21 ; CCN(CC)CCCn1c2ccccc2c2cc(C(=O)N3CCN(CCc4cccc(OC)c4)CC3)ccc21]

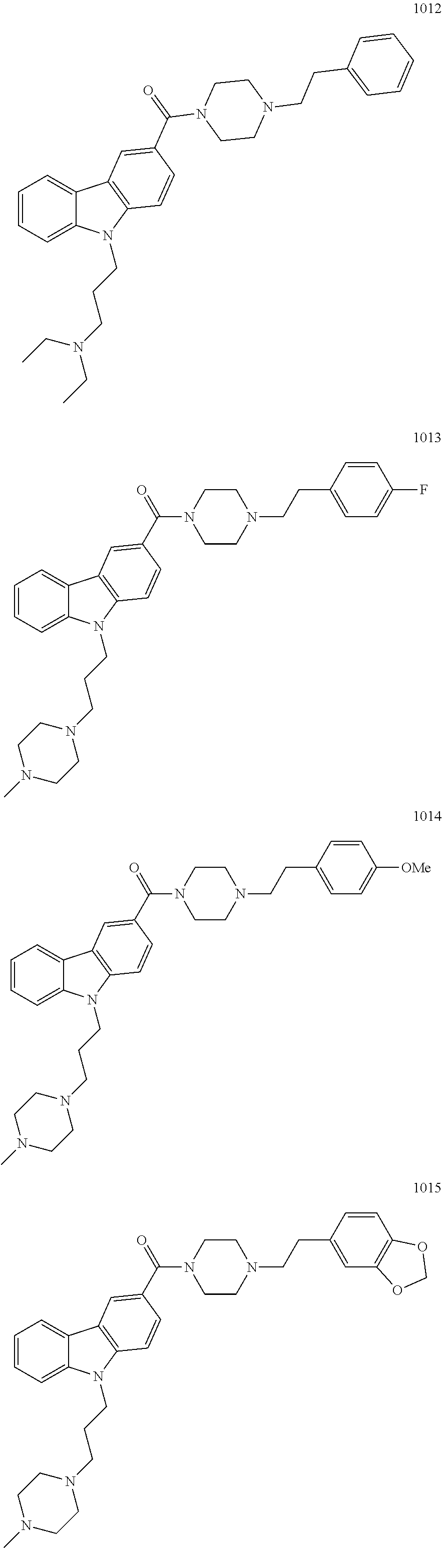 [SMILES: CCN(CC)CCCn1c2ccccc2c2cc(C(=O)N3CCN(CCc4ccccc4)CC3)ccc21 ; CN1CCN(CCCn2c3ccccc3c3cc(C(=O)N4CCN(CCc5ccc(F)cc5)CC4)ccc32)CC1 ; COc1ccc(CCN2CCN(C(=O)c3ccc4c(c3)c3ccccc3n4CCCN3CCN(C)CC3)CC2)cc1 ; CN1CCN(CCCn2c3ccccc3c3cc(C(=O)N4CCN(CCc5ccc6c(c5)OCO6)CC4)ccc32)CC1]

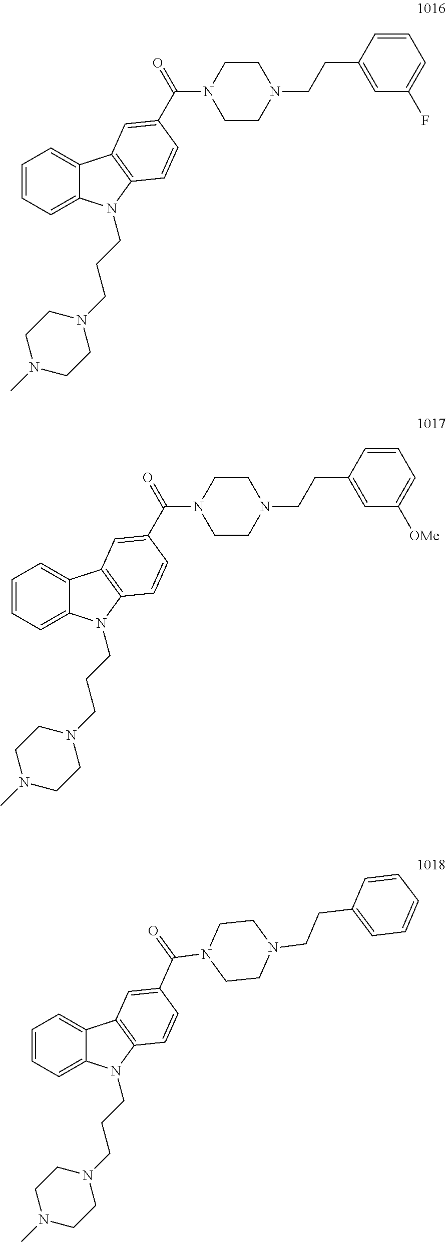 [SMILES: CN1CCN(CCCn2c3ccccc3c3cc(C(=O)N4CCN(CCc5cccc(F)c5)CC4)ccc32)CC1 ; COc1cccc(CCN2CCN(C(=O)c3ccc4c(c3)c3ccccc3n4CCCN3CCN(C)CC3)CC2)c1 ; CN1CCN(CCCn2c3ccccc3c3cc(C(=O)N4CCN(CCc5ccccc5)CC4)ccc32)CC1]

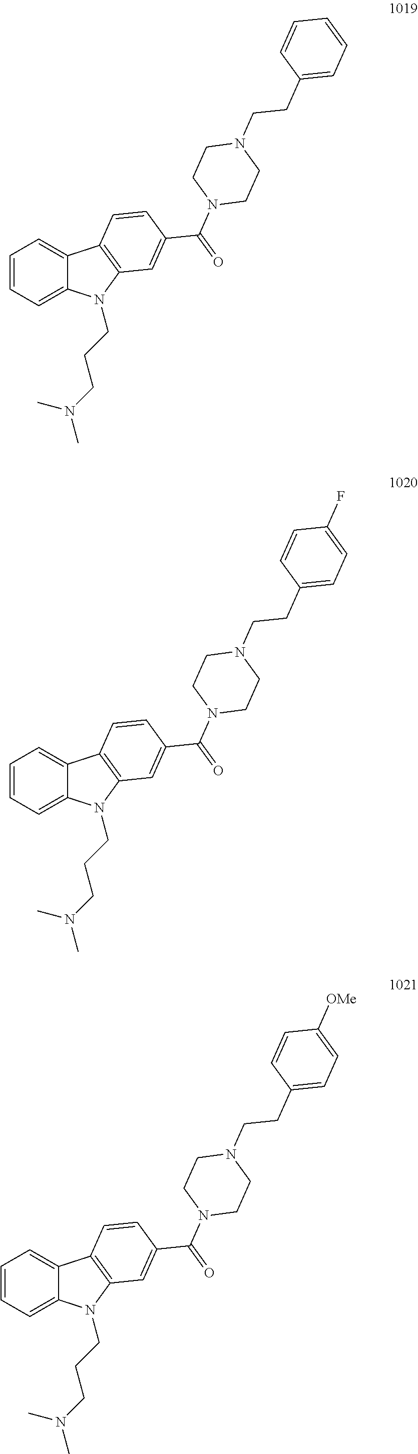 [SMILES: CN(C)CCCn1c2ccccc2c2ccc(C(=O)N3CCN(CCc4ccccc4)CC3)cc21 ; CN(C)CCCn1c2ccccc2c2ccc(C(=O)N3CCN(CCc4ccc(F)cc4)CC3)cc21 ; COc1ccc(CCN2CCN(C(=O)c3ccc4c5ccccc5n(CCCN(C)C)c4c3)CC2)cc1]

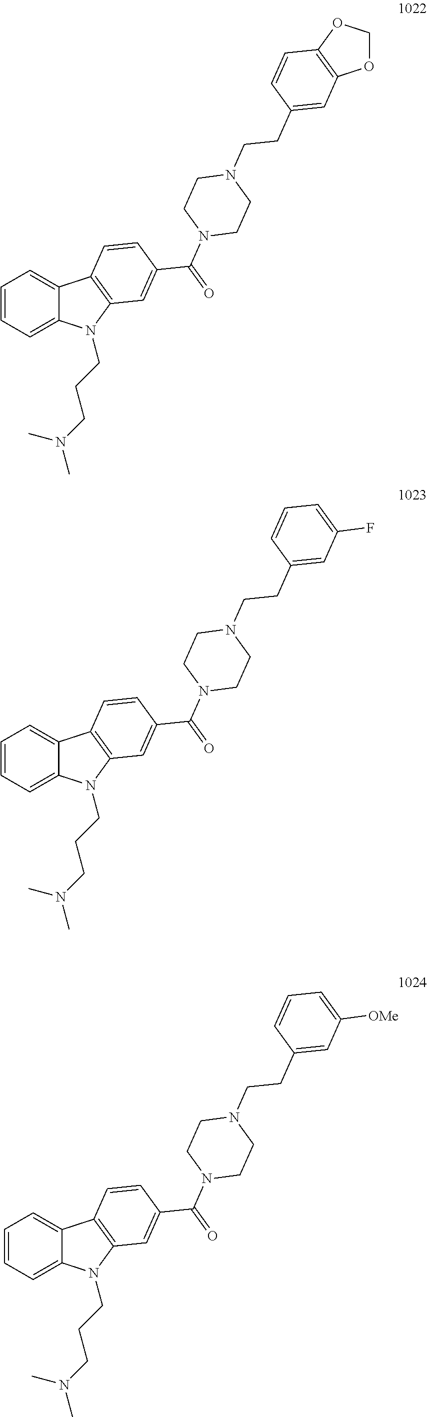 [SMILES: CN(C)CCCn1c2ccccc2c2ccc(C(=O)N3CCN(CCc4ccc5c(c4)OCO5)CC3)cc21 ; CN(C)CCCn1c2ccccc2c2ccc(C(=O)N3CCN(CCc4cccc(F)c4)CC3)cc21 ; COc1cccc(CCN2CCN(C(=O)c3ccc4c5ccccc5n(CCCN(C)C)c4c3)CC2)c1]

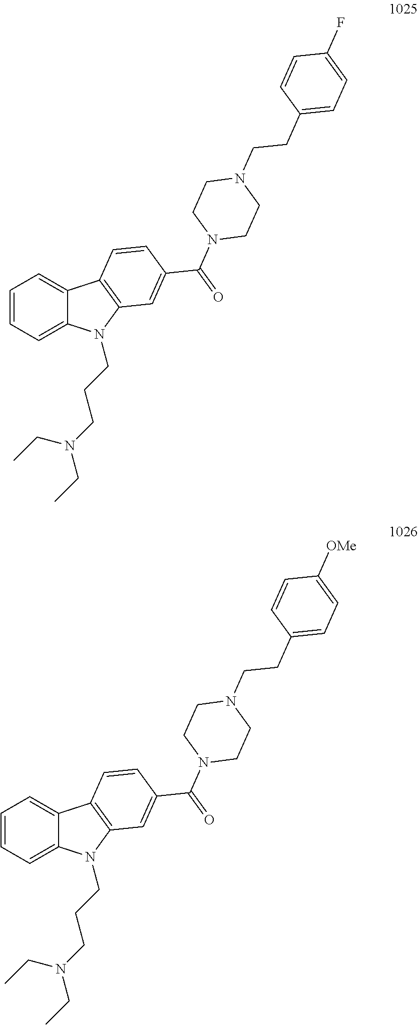 [SMILES: CCN(CC)CCCn1c2ccccc2c2ccc(C(=O)N3CCN(CCc4ccc(F)cc4)CC3)cc21 ; CCN(CC)CCCn1c2ccccc2c2ccc(C(=O)N3CCN(CCc4ccc(OC)cc4)CC3)cc21]

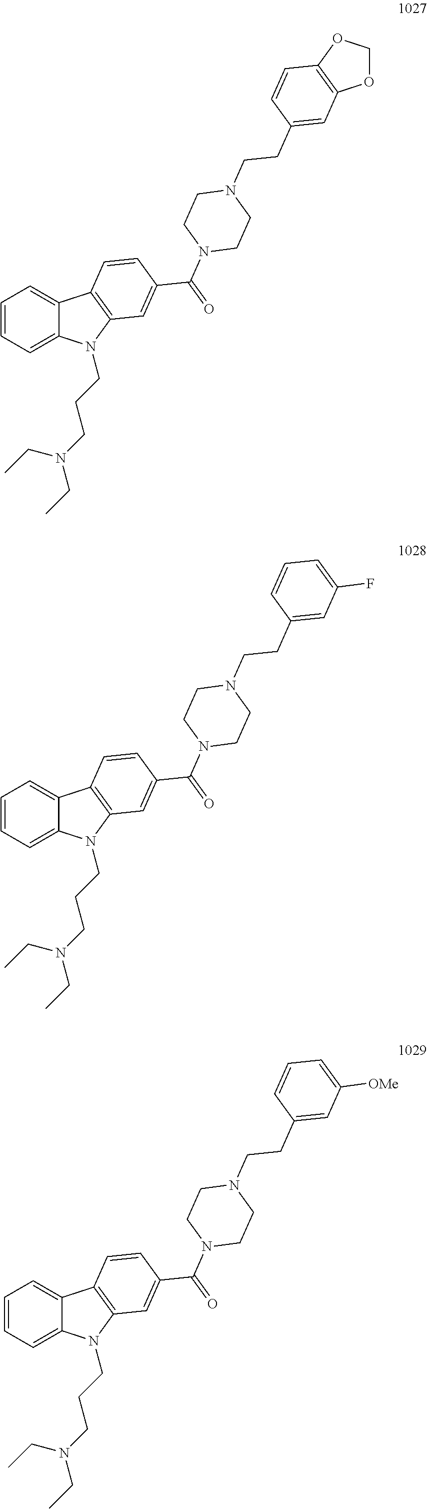 [SMILES: CCN(CC)CCCn1c2ccccc2c2ccc(C(=O)N3CCN(CCc4ccc5c(c4)OCO5)CC3)cc21 ; CCN(CC)CCCn1c2ccccc2c2ccc(C(=O)N3CCN(CCc4cccc(F)c4)CC3)cc21 ; CCN(CC)CCCn1c2ccccc2c2ccc(C(=O)N3CCN(CCc4cccc(OC)c4)CC3)cc21]

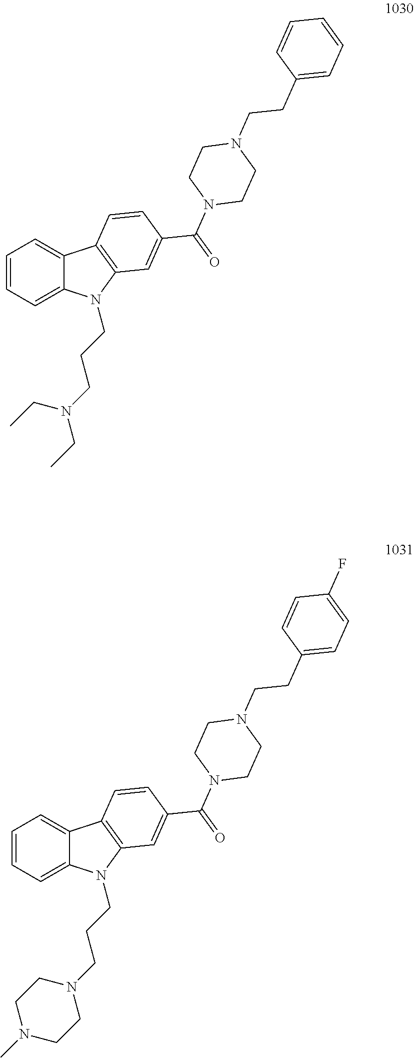 [SMILES: CCN(CC)CCCn1c2ccccc2c2ccc(C(=O)N3CCN(CCc4ccccc4)CC3)cc21 ; CN1CCN(CCCn2c3ccccc3c3ccc(C(=O)N4CCN(CCc5ccc(F)cc5)CC4)cc32)CC1]

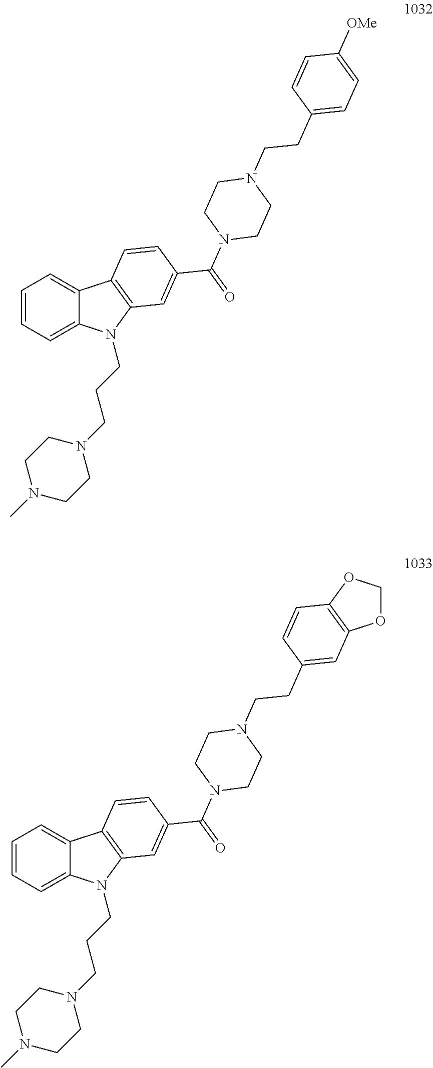 [SMILES: COc1ccc(CCN2CCN(C(=O)c3ccc4c5ccccc5n(CCCN5CCN(C)CC5)c4c3)CC2)cc1 ; CN1CCN(CCCn2c3ccccc3c3ccc(C(=O)N4CCN(CCc5ccc6c(c5)OCO6)CC4)cc32)CC1]

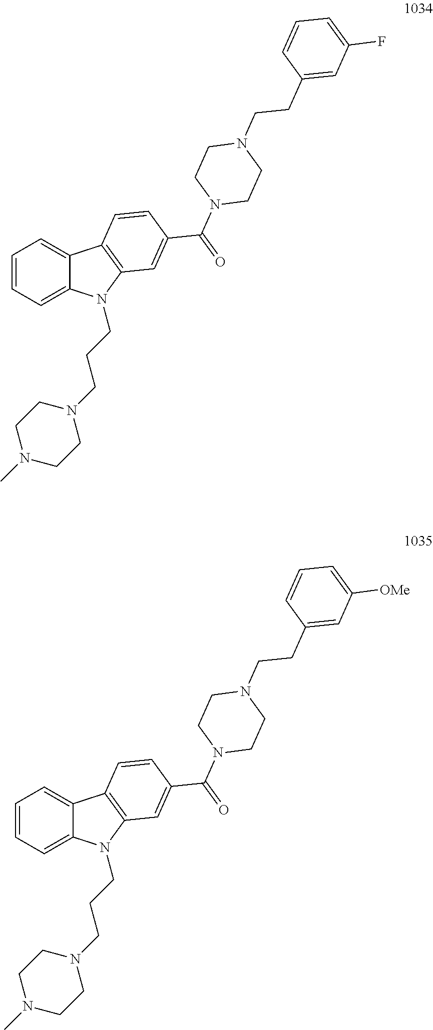 [SMILES: CN1CCN(CCCn2c3ccccc3c3ccc(C(=O)N4CCN(CCc5cccc(F)c5)CC4)cc32)CC1 ; COc1cccc(CCN2CCN(C(=O)c3ccc4c5ccccc5n(CCCN5CCN(C)CC5)c4c3)CC2)c1]

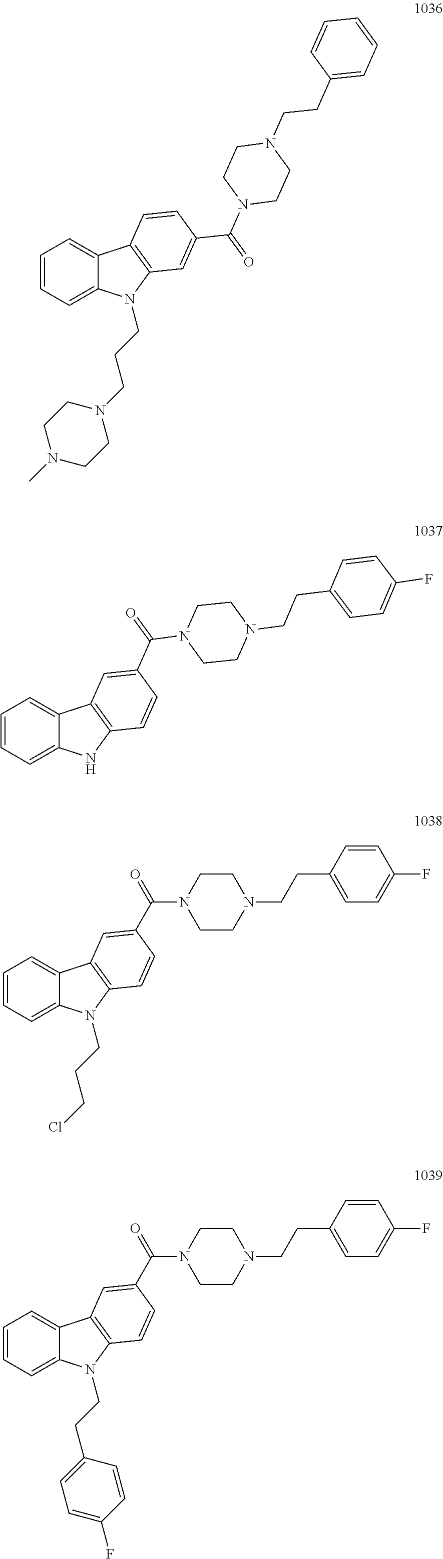 [SMILES: CN1CCN(CCCn2c3ccccc3c3ccc(C(=O)N4CCN(CCc5ccccc5)CC4)cc32)CC1 ; O=C(c1ccc2[nH]c3ccccc3c2c1)N1CCN(CCc2ccc(F)cc2)CC1 ; O=C(c1ccc2c(c1)c1ccccc1n2CCCCl)N1CCN(CCc2ccc(F)cc2)CC1 ; O=C(c1ccc2c(c1)c1ccccc1n2CCc1ccc(F)cc1)N1CCN(CCc2ccc(F)cc2)CC1]

In one embodiment, the compounds are:

[3-(3-{4-[2-(4-fluorophenyl)ethyl]piperazine-1-carbonyl}-9H-carbazol-9-yl)propyl]dimethylamine;

[3-(3-{4-[2-(4-methoxyphenyl)ethyl]piperazine-1-carbonyl}-9H-carbazol-9-yl)propyl]dimethylamine;

[3-(3-{4-[2-(2H-1,3-benzodioxol-5-yl)ethyl]piperazine-1-carbonyl}-9H-carbazol-9-yl)propyl]dimethylamine;

[3-(3-{4-[2-(3-fluorophenyl)ethyl]piperazine-1-carbonyl}-9H-carbazol-9-yl)propyl]dimethylamine;

[3-(3-{4-[2-(3-methoxyphenyl)ethyl]piperazine-1-carbonyl}-9H-carbazol-9-yl)propyl]dimethylamine;

dimethyl(3-{3-[4-(2-phenylethyl)piperazine-1-carbonyl]-9H-carbazol-9-yl}propyl)amine;

diethyl[3-(3-{4-[2-(4-fluorophenyl)ethyl]piperazine-1-carbonyl}-9H-carbazol-9-yl)propyl]amine;

diethyl[3-(3-{4-[2-(4-methoxyphenyl)ethyl]piperazine-1-carbonyl}-9H-carbazol-9-yl)propyl]amine;

[3-(3-{4-[2-(2H-1,3-benzodioxol-5-yl)ethyl]piperazine-1-carbonyl}-9H-carbazol-9-yl)propyl]diethylamine;

diethyl[3-(3-{4-[2-(3-fluorophenyl)ethyl]piperazine-1-carbonyl}-9H-carbazol-9-yl)propyl]amine;

diethyl[3-(3-{4-[2-(3-methoxyphenyl)ethyl]piperazine-1-carbonyl}-9H-carbazol-9-yl)propyl]amine;

diethyl(3-{3-[4-(2-phenylethyl)piperazine-1-carbonyl]-9H-carbazol-9-yl}propyl)amine;

3-{4-[2-(4-fluorophenyl)ethyl]piperazine-1-carbonyl}-9-[3-(4-methylpiperazin-1-yl)propyl]-9H-carbazole;

3-{4-[2-(4-methoxyphenyl)ethyl]piperazine-1-carbonyl}-9-[3-(4-methylpiperazin-1-yl)propyl]-9H-carbazole;

3-{4-[2-(2H-1,3-benzodioxol-5-yl)ethyl]piperazine-1-carbonyl}-9-[3-(4-methylpiperazin-1-yl)propyl]-9H-carbazole;

3-{4-[2-(3-fluorophenyl)ethyl]piperazine-1-carbonyl}-9-[3-(4-methylpiperazin-1-yl)propyl]-9H-carbazole;

3-{4-[2-(3-methoxyphenyl)ethyl]piperazine-1-carbonyl}-9-[3-(4-methylpiperazin-1-yl)propyl]-9H-carbazole;

9-[3-(4-methylpiperazin-1-yl)propyl]-3-[4-(2-phenylethyl)piperazine-1-carbonyl]-9H-carbazole;

[3-(2-{4-[2-(4-fluorophenyl)ethyl]piperazine-1-carbonyl}-9H-carbazol-9-yl)propyl]dimethylamine;

[3-(2-{4-[2-(4-methoxyphenyl)ethyl]piperazine-1-carbonyl}-9H-carbazol-9-yl)propyl]dimethylamine;

[3-(2-{4-[2-(2H-1,3-benzodioxol-5-yl)ethyl]piperazine-1-carbonyl}-9H-carbazol-9-yl)propyl]dimethylamine;

[3-(2-{4-[2-(3-fluorophenyl)ethyl]piperazine-1-carbonyl}-9H-carbazol-9-yl)propyl]dimethylamine;

[3-(2-{4-[2-(3-methoxyphenyl)ethyl]piperazine-1-carbonyl}-9H-carbazol-9-yl)propyl]dimethylamine;

dimethyl(3-{2-[4-(2-phenylethyl)piperazine-1-carbonyl]-9H-carbazol-9-yl}propyl)amine;

diethyl[3-(2-{4-[2-(4-fluorophenyl)ethyl]piperazine-1-carbonyl}-9H-carbazol-9-yl)propyl]amine;

diethyl[3-(2-{4-[2-(4-methoxyphenyl)ethyl]piperazine-1-carbonyl}-9H-carbazol-9-yl)propyl]amine;

[3-(2-{4-[2-(2H-1,3-benzodioxol-5-yl)ethyl]piperazine-1-carbonyl}-9H-carbazol-9-yl)propyl]diethylamine;

diethyl[3-(2-{4-[2-(3-fluorophenyl)ethyl]piperazine-1-carbonyl}-9H-carbazol-9-yl)propyl]amine

diethyl[3-(2-{4-[2-(3-methoxyphenyl)ethyl]piperazine-1-carbonyl}-9H-carbazol-9-yl)propyl]amine;

diethyl(3-{2-[4-(2-phenylethyl)piperazine-1-carbonyl]-9H-carbazol-9-yl}propyl)amine;

2-{4-[2-(4-fluorophenyl)ethyl]piperazine-1-carbonyl}-9-[3-(4-methylpiperazin-1-yl)propyl]-9H-carbazole;

2-{4-[2-(4-methoxyphenyl)ethyl]piperazine-1-carbonyl}-9-[3-(4-methylpiperazin-1-yl)propyl]-9H-carbazole;

2-{4-[2-(2H-1,3-benzodioxol-5-yl)ethyl]piperazine-1-carbonyl}-9-[3-(4-methylpiperazin-1-yl)propyl]-9H-carbazole;

2-{4-[2-(3-fluorophenyl)ethyl]piperazine-1-carbonyl}-9-[3-(4-methylpiperazin-1-yl)propyl]-9H-carbazole;

2-{4-[2-(3-methoxyphenyl)ethyl]piperazine-1-carbonyl}-9-[3-(4-methylpiperazin-1-yl)propyl]-9H-carbazole;

9-[3-(4-methylpiperazin-1-yl)propyl]-2-[4-(2-phenylethyl)piperazine-1-carbonyl]-9H-carbazole;

(9H-carbazol-3-yl)(4-(4-fluorophenethyl)piperazin-1-yl)methanone;

(9-(3-chloropropyl)-9H-carbazol-3-yl)(4-(4-fluorophenethyl)piperazin-1-yl)methanone;

(9-(4-fluorophenethyl)-9H-carbazol-3-yl)(4-(4-fluorophenethyl)piperazin-1-yl)methanone.

In a second aspect the invention relates to a pharmaceutical composition comprising a compound of formula (I) together with a pharmaceutically acceptable carrier, diluent or excipient.

Compounds and pharmaceutical compositions according to the present invention may be suitable for the treatment or prevention of a proliferative disease. Accordingly, in another aspect the invention relates to a method of treating or preventing a proliferative disease in a subject, the method comprising administering to the subject an effective amount of a compound of formula (I) according to the first aspect of the invention or a pharmaceutical composition according to the second aspect of the invention.

In a further aspect the present invention relates to the use of a compound of formula (I) according to the first aspect of the invention or a pharmaceutical composition according to the second aspect of the invention in the manufacture of a medicament for treating or preventing a proliferative disease.

In a further aspect the present invention relates to the use of a compound of formula (I) according to the first aspect of the invention or a pharmaceutical composition according to the second aspect of the invention for the treatment or prevention of a proliferative disease in a subject.

In a further aspect the present invention relates to a compound of formula (I) according to the first aspect of the invention or a pharmaceutical composition according to the second aspect of the invention for use in the treatment or prevention of a proliferative disease in a subject.

In one or more preferred embodiments, the proliferative disease is cancer, preferably a solid tumour. In various preferred embodiments, the cancer is selected from the group consisting of breast cancer, lung cancer, prostate cancer, ovarian cancer, uterine cancer brain cancer, skin cancer, colon cancer and bladder cancer.

Those skilled in the art will understand that in the context of the present invention an ‘effective amount’ is an amount sufficient to produce a desired therapeutic or pharmacological effect in the subject being treated.

In a further aspect the invention relates to a method of completely or partially preventing the recurrence of a solid tumour in a subject, the method comprising administering to the subject an effective amount of a compound of formula (I) according to the first aspect of the invention or a pharmaceutical composition according to the second aspect of the invention.

In another aspect the invention relates to the use of a compound according to the first aspect of the invention or the pharmaceutical composition according to the second aspect of the invention in the manufacture of a medicament for completely or partially preventing the recurrence of a solid tumour.

In a further aspect the present invention relates to the use of a compound of formula (I) according to the first aspect of the invention or a pharmaceutical composition according to the second aspect of the invention for completely or partially preventing the recurrence of a solid tumour in a subject.

In a further aspect the present invention relates to a compound of formula (I) according to the first aspect of the invention or a pharmaceutical composition according to the second aspect of the invention for use in completely or partially preventing the recurrence of a solid tumour in a subject.

Compounds and pharmaceutical compositions according to the present invention may be suitable for the treatment or prevention of an inflammatory disease or disorder. Accordingly, in another aspect the invention relates to a method of treating an inflammatory disease or disorder in a subject, the method comprising administering to the subject an effective amount of a compound of formula (I) according to the first aspect of the invention or a pharmaceutical composition according to the second aspect of the invention.

In a further aspect the present invention relates to the use of a compound of formula (I) according to the first aspect of the invention or a pharmaceutical composition according to the second aspect of the invention in the manufacture of a medicament for treating an inflammatory disease or disorder.

In a further aspect the present invention relates to the use of a compound of formula (I) according to the first aspect of the invention or a pharmaceutical composition according to the second aspect of the invention for the treatment of an inflammatory disease or disorder in a subject.

In a further aspect the present invention relates to a compound of formula (I) according to the first aspect of the invention or a pharmaceutical composition according to the second aspect of the invention for use in the treatment of an inflammatory disease or disorder in a subject.

In one or more preferred embodiments, the inflammatory disease or disorder is selected from osteoarthritis, inflammatory bowel disease (e.g. ulcerative colitis and Crohn's disease), ulcerative proctitis, distal colitis, autoimmune disorders (e.g. SLE, rheumatoid arthritis, glomerulonephritis), asthma and diseases involving pulmonary inflammation, and cardiovascular disorders (e.g. atherosclerosis, hypertension and lipid dyscrasias).

The compounds of formula (I) may be used in therapy alone or in combination with one or more other agents (e.g. chemotherapeutic or anti-inflammatory agents), for example, as part of a combination therapy.

In another aspect the present invention relates to a process for preparing a compound of formula (I) comprising the steps of:

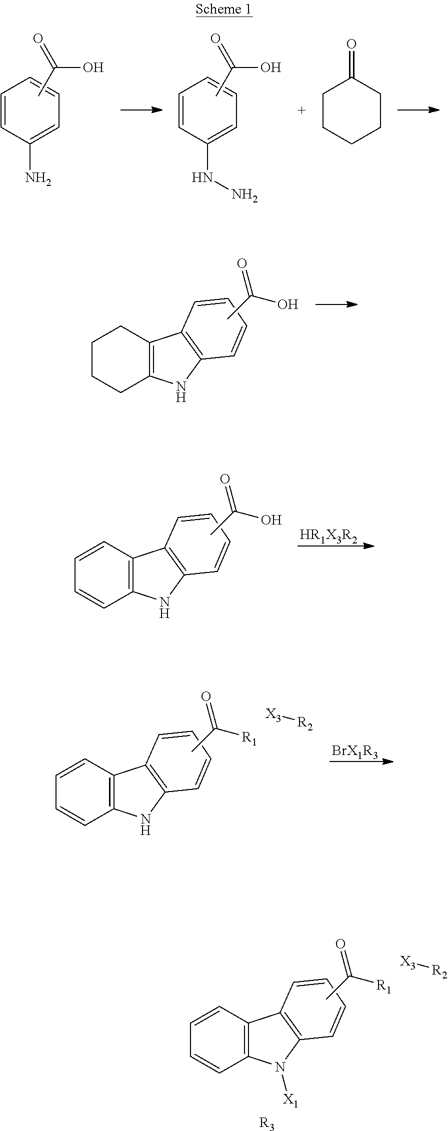

Further aspects of the present invention and further embodiments of the aspects described in the preceding paragraphs will become apparent from the following description, given by way of example and with reference to the accompanying drawings.

### DESCRIPTION OF THE DRAWINGS

FIG. 1: Imaging and quantitation of actin filaments in SK-N-SH neuroblastoma cells treated with compound (A) 1007, (B) 1013 and (C) 1016. Cells were stained with 488-Atto-Phallodin and DAPI to visualize the actin filament bundles and the nucleus, respectively. Shown in the top panel is a representative gray scale immunofluorescent image from control (vehicle alone), 5 μM and 10 μM treated cells. The middle panel (enlarged inset bottom panel) shows the overlay of the cell image with the linear feature quantitation. The coloured lines indicate the detected actin filaments. Also shown is the quantitation of cell number, filament number/cell and filament number/cell area (μM²). Statistical analysis was performed using a one way ANNOVA-multiple comparison where each drug treated group was compared to the control. ****p<0.0001, ****p<0.001, ***p<0.01, **p<0.1.

FIG. 2: Imaging and quantitation of Tpm3.1-containing actin filaments in SK-N-SH neuroblastoma cells treated with compound (A) 1007, (B) 1013 and (C) 1016. Cells were stained with γ9d (sheep polycolonal, 1:100) followed by 488-conjugated secondary (1:1000) and DAPI to visualize the Tpm3.1 containing filament bundles and the nucleus, respectively. Shown in the top panel is a representative gray scale immunofluorescent image from control (vehicle alone), 5 μM and 7.5 μM treated cells. The middle panel (enlarged inset bottom panel) shows the overlay of the cell image with the linear feature quantitation. The coloured lines indicate the detected Tpm3.1 containing actin filaments. Also shown is the quantitation of cell number, Tpm3.1 filament number/cell and Tpm3.1 filament number/cell area (μM²). Statistical analysis was performed using a one way ANNOVA-multiple comparison where each drug treated group was compared to the control. ****p<0.0001.

FIG. 3: Impact of compound 1001 on the polymerization of tropomyosin and its subsequent association with actin. Increasing concentrations of (A) cytoskeletal Tpm3.1 or (B) muscle alpha-fast Tm (0.3-4.0 μM) were pre-incubated with 1% (v/v) DMSO (vehicle control) or 50 μM ATM-1001 prior to combining with 3 μM F-actin. The Tm/F-actin mixture was sedimented and the ratio of tropomyosin:actin in the pellet, normalized to the ratio that occurs at tropomyosin saturation, was plotted against the concentration of unbound tropomyosin in the supernatant.

FIG. 4: Effect of anti-tropomyosin compounds on actin-activated myosin II ATPase activity. Skeletal myosin II (30-60 nM) was incubated with 1% (v/v) DMSO or 50 μM of Blebbistatin, TR100 or compound 1001 for 5 min prior to mixing with 3 μM F-actin. Reactions were initiated with 1 mM ATP and inorganic phosphate release was detected spectrophotometrically. Data represents mean±SEM, for n=2-4 independent repeats. **P-value, 0.001<0.01.

FIG. 5: Effect of compound 1001 on tumour growth in vivo. Compound 1001 was administered IV at 60 mg/kg for 16 days in a flank xenograft model of melanoma (A375). Tumour volume was measured every 2-3 days. **p<0.01, ***P<0.001.

### DETAILED DESCRIPTION

The invention is based on the surprising finding that compounds of general formula (I) effectively inhibit tropomyosin, which results in unexpected improvement in the treatment of proliferative diseases, particularly cancer. The development of the actin cytoskeleton involves a number of ancillary control and regulatory proteins. Identification and specific targeting of actin regulatory proteins associated with the cytoskeleton of cancer cells offers the opportunity to develop cancer specific drugs without unwanted side effects.

Actin filaments are constructed through the polymerisation of globular actin protein monomers. The actin monomer is polar, with one end bearing a positive charge and the other end a negative charge. The actin filaments thus have all the actin proteins aligned in one direction. These filaments have secondary coiled proteins, tropomyosins, associated with them. The tropomyosins play an integral role in regulating the function of actin filaments. Structurally the actin filaments are made up of polymeric actin monomers with tropomyosin dimers sitting in the alpha helical groove of the actin filament to form a homopolymer. There are more than 40 mammalian tropomyosin isoforms, each of which regulates specific actin filaments. There are specific isoforms of tropomyosins that regulate the cytoskeleton of cancer cells; disruption of this interaction offers a basis to specifically treat cancer cells.

I. Definitions

The following are some definitions of terms used in the art that may be helpful in understanding the description of the present invention. These are intended as general definitions and should in no way limit the scope of the present invention to those terms alone, but are put forth for a better understanding of the following description.

Unless the context requires otherwise or specifically states to the contrary, integers, steps, or elements of the invention recited herein as singular integers, steps or elements clearly encompass both singular and plural forms of the recited integers, steps or elements.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps, features, compositions and compounds.

The terms “comprising” and “including” are used herein in their open-ended and non-limiting sense unless otherwise noted.

The term “optionally substituted” as used throughout the specification denotes that the group may or may not be further substituted or fused (so as to form a polycyclic system), with one or more non-hydrogen substituent groups. Suitable chemically viable optional substituents for a particular functional group will be apparent to those skilled in the art. Typical optional substituents include C₁-C₄ alkyl, C₂-C₄ alkenyl, OH, halogen, O(C₁-C₄ alkyl), NR^{a}R^{b} wherein R^{a} and R^{b} are independently selected from H, C₁-C₃ alkyl, CONH₂, SH, S(C₁-C₃ alkyl), —CH₂—O(C₁₋₃ alkyl), C₆₋₁₀ aryl, —CH₂-phenyl, hydroxyl-(C₁₋₃ alkyl), and halo-(C₁₋₃ alkyl). Presently preferred optional substituents include C₁₋₃ alkyl, C₁₋₃ alkoxy, —CH₂—(C₁₋₃)alkoxy, C₆₋₁₀ aryl, —CH₂-phenyl, halogen, OH, hydroxy-(C₁₋₃)alkyl, and halo-(C₁₋₃)alkyl, e.g, CF₃, CH₂CF₃.

“Acyl” means an alkyl-CO— group in which the alkyl group is as described herein. Examples of acyl include acetyl and benzoyl. The alkyl group may be a C₁-C₆ alkyl, C₁-C₄ alkyl, or C₁-C₃ alkyl group. The group may be a terminal group or a bridging group.

“Alkyl” as a group or part of a group refers to a straight or branched aliphatic hydrocarbon group having 1-12 carbon atoms, or 1-10 carbon atoms, or 1-6 carbon atoms, or 1-4 carbon atoms, or 1-3 carbon atoms. Thus, for example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethyl butyl, 1,1,3-trimethylbutyl, 5-methylheptyl, 1-methylheptyl, octyl, nonyl, decyl, and the like. The group may be a terminal group or a bridging group.

“Alkenyl” as a group or part of a group denotes an aliphatic hydrocarbon group containing at least one carbon-carbon double bond and which may be straight or branched such as a group having 2-12 carbon atoms, or 2-6 carbon atoms, or 2-4 carbon atoms, in the normal chain. The group may contain a plurality of double bonds in the normal chain and the orientation about each double bond is independently cis or trans, E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like. The group may be a terminal group or a bridging group.

“Alkenyloxy” refers to an —O— alkenyl group in which alkenyl is as defined herein. Preferred alkenyloxy groups are C₂-C₁₂ alkenyloxy groups. The group may be a terminal group or a bridging group.

The terms “alkyloxy” and “alkoxy” are synonymous and refer to an —O-alkyl group in which alkyl is defined herein. Presently preferred alkoxy groups are C₁₋₆ alkoxy or C₁₋₄ alkoxy or C₁₋₃ alkoxy. Examples include, but are not limited to, methoxy, ethoxy, n-propoxy, isopropoxy, sec-butoxy, tert-butoxy, and the like. The group may be a terminal group or a bridging group.

“Alkylamino” includes both mono-alkylamino and dialkylamino, unless specified. “Mono-alkylamino” means a —NH-alkyl group, in which alkyl is as defined above. “Dialkylamino” means a —N(alkyl)₂ group, in which each alkyl may be the same or different and are each as defined herein for alkyl. The alkyl group may be a C₁-C₆ alkyl group. The group may be a terminal group or a bridging group.

“Alkynyl” as a group or part of a group means an aliphatic hydrocarbon group containing a carbon-carbon triple bond and which may be straight or branched and may have from 2-12 carbon atoms or 2-6 carbon atoms or 2-4 carbon atoms in the normal chain. Exemplary structures include, but are not limited to, ethynyl and propynyl. The group may be a terminal group or a bridging group.

“Alkynyloxy” refers to an —O-alkynyl group in which alkynyl is as defined herein. Presently preferred alkynyloxy groups are C₂-C₆ alkynyloxy groups, C₂-C₄ alkynyloxy. The group may be a terminal group or a bridging group.

“Aryl” as a group or part of a group denotes (i) an optionally substituted monocyclic, or fused polycyclic, aromatic carbocycle (ring structure having ring atoms that are all carbon) that may have from 5-18 atoms per ring. Presently preferred aryl groups have 6-14 atoms per ring, or more preferably 6-10 atoms per ring. Examples of aryl groups include phenyl, naphthyl, phenanthryl and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group.

“Cycloalkenyl” means a non-aromatic monocyclic or multicyclic ring system containing at least one carbon-carbon double bond and may have from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group.

“Cycloalkyl” refers to a saturated or partially saturated, monocyclic or fused or spiro polycyclic, carbocycle that may contain from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

The terms “halogen” or “halo” are synonymous and refer to fluorine, chlorine, bromine or iodine.

“Heteroaryl” either alone or as part of a group refers to groups containing an aromatic ring (such as a 5- or 6-membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

The term “carbocyclic ring” as used herein refers to a carbon-based ring system. It is intended to include aryl, cycloalkenyl, cycloalkyl, and heteroaryl groups, as defined herein.

The term “heteroatom” or variants such as “hetero-” as used herein refers to O, N, NH and S.

Certain compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, formula (I) is intended to cover, where applicable, solvated as well as unsolvated forms of the compounds. Thus, formula (I) includes compounds having the indicated structure, including the hydrated or solvated form, as well as the non-hydrated and non-solvated forms.

The term “pharmaceutically acceptable salt” refers to those salts which, within the scope of sound medical judgment, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, mandelic, ambonic, pamoic, pantothenic, sulfanilic, cyclohexylaminosulfonic, stearic, algenic, p-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of the present invention. For example an ester prodrug of a compound of the present invention containing a hydroxyl group may be convertible by hydrolysis in vivo to the parent molecule. Suitable esters are for example, acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of cancer or its associated symptoms.

“Preventing” or “prevention” means preventing the occurrence of the cancer or tempering the severity of the cancer if it develops subsequent to the administration of the compounds or pharmaceutical compositions of the present invention. This prevents the onset of clinically evident unwanted cell proliferation altogether or the onset of a pre-clinically evident stage of unwanted rapid cell proliferation in individuals at risk. Also intended to be encompassed by this definition is the prevention of metastases of malignant cells or the arrest or reversal of the progression of malignant cells.

The terms “therapeutically effective” or “pharmacologically effective” are intended to qualify the amount of each agent which will achieve the goal of improvement in disease severity and the frequency of incidence over treatment of each agent by itself while avoiding adverse side effects typically associated with other therapies.

A “pharmaceutical carrier, diluent or excipient” includes, but is not limited to, any physiological buffered (i.e., about pH 7.0 to 7.4) medium comprising a suitable water soluble organic carrier, conventional solvents, dispersion media, fillers, solid carriers, coatings, antibacterial and antifungal agents, isotonic and absorption delaying agents. Suitable water soluble organic carriers include, but are not limited to saline, dextrose, corn oil, dimethylsulfoxide, and gelatin capsules. Other conventional additives include lactose, mannitol, corn starch, potato starch, binders such as crystalline cellulose, cellulose derivatives, acacia, gelatins, disintegrators such as sodium carboxymethyl-cellulose, and lubricants such as talc or magnesium stearate.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

In the context of this specification the term “administering” and variations of that term including “administer” and “administration”, includes contacting, applying, delivering or providing a compound or composition of the invention to an organism, or a surface by any appropriate means.

II. Synthesis of Compounds of the Invention

The present invention relates to functionalized carbazole compounds of general formula (I) as defined herein, and to the use of such compounds as therapeutic agents.

Compounds of general formula (I), or salts, hydrates or solvates, thereof may be prepared by methods known to those skilled in the art. The general synthetic scheme for preparing compounds of formula (I) is described below:

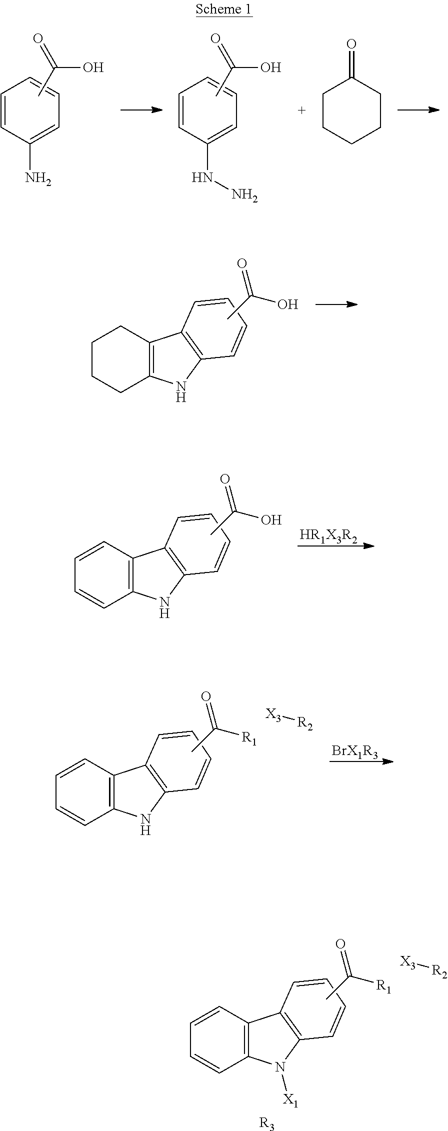

The methods described above in Scheme 1 may offer one or more advantages including high yields, control of stereochemistry, few synthetic steps and reaction conditions that are amenable to large scale manufacture.

The methods described above are merely representative and routine modifications and variations that would be apparent to persons skilled in the art fall within the broad scope and ambit of the invention disclosed herein.

III. Methods of Treatment using Compounds of the Invention

The compounds of general formula (I) according to the present invention, and pharmaceutical compositions thereof, may be used in the treatment or prevention of proliferative diseases, preferably cancer. The compounds and compositions of the invention may be useful for the treatment of a wide variety of cancers (tumours), including but not limited to, solid tumours, such as for example, breast cancer, lung cancer, prostate cancer, ovarian cancer, uterine cancer brain cancer, skin cancer, colon cancer and bladder cancer.

Advantageously, compounds of the present invention may possess superior pharmaceutical properties, such as improved resistance to conjugation via glucuronyl transferases and other water solubilizing transferases such as sulfases, which may be over-expressed on proliferative cells such as cancer cells. This may advantageously confer superior pharmaceutical properties, such as an enhanced pharmacokinetic profile through reduced conjugation and elimination.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds or pharmaceutical compositions of the present invention may be administered orally, intravenously, intranasally, rectally, parenterally, subcutaneously, intramuscularly, topically or by any means which delivers an effective amount of the active agent to the tissue or site to be treated. It will be appreciated that different dosages may be required for treating different disorders. An effective amount of an agent is that amount which causes a statistically significant decrease in neoplastic cell count, growth, or size. Neoplastic disorders responsive to the agents of the present invention include, but are not limited to, breast cancer.

The dosage form and amount of the compounds or pharmaceutical compositions of the present invention can be readily established by reference to known treatment or prophylactic regimens.

For example, the compounds and pharmaceutical compositions may be formulated for oral, injectable, rectal, parenteral, subcutaneous, intravenous or intramuscular delivery. Non-limiting examples of particular formulation types include tablets, capsules, caplets, powders, granules, injectables, ampoules, vials, ready-to-use solutions or suspensions, lyophilized materials, suppositories and implants. The solid formulations such as the tablets or capsules may contain any number of suitable pharmaceutically acceptable excipients or carriers described above.

For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. Suitable formulations may include cyclodextrins (e.g. sulfobutyl-ether-beta-cyclodextrin, or SBECD, commercially-available as Dexolve, or the formulation aid known as Captisol). The formulations may be present in unit or multi-dose containers such as sealed ampoules or vials.

The amount of therapeutically effective compound that is administered and the dosage regimen for treating a disease condition with the compounds and/or pharmaceutical compositions of the invention depends on a variety of factors, including the age, weight, sex, and medical condition of the subject, the severity of the disease, the route and frequency of administration, the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

The compounds of the present invention may be administered along with a pharmaceutical carrier, diluent or excipient as described above. Alternatively, or in addition to, the compounds may be administered in combination with other agents, for example, chemotherapeutic or immune-stimulating drugs or therapeutic agents.

The terms “combination therapy” or “adjunct therapy” in defining use of a compound of the present invention and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments of the present invention one or more compounds of general formula (I) may be formulated or administered in combination with one or more other therapeutic agents. Thus, in accordance with various embodiments of the present invention, one or more compounds of general formula (I) may be included in combination treatment regimens with surgery and/or other known treatments or therapeutic agents, such as other anticancer agents, in particular, chemotherapeutic agents, radiotherapeutic agents, and/or adjuvant or prophylactic agents.

There are large numbers of antineoplastic agents available in commercial use, in clinical evaluation and in pre-clinical development, which could be selected for treatment of cancers or other neoplasias by combination drug chemotherapy. Such anti-neoplastic agents fall into several major categories, namely, antibiotic-type agents, alkylating agents, antimetabolite agents, hormonal agents, immunological agents, interferon-type agents and a category of miscellaneous agents. Alternatively, other anti-neoplastic agents, such as metallomatrix proteases inhibitors may be used. Suitable agents which may be used in combination therapy will be recognized by those of skill in the art. Suitable agents are listed, for example, in the Merck Index, An Encyclopaedia of Chemicals, Drugs and Biologicals, 12^{th} Ed., 1996, the entire contents of which are incorporated herein by reference.

Combination regimens may involve the active agents being administered together, sequentially, or spaced apart as appropriate in each case. Combinations of active agents including compounds of the invention may be synergistic.

The co-administration of compounds of the general formula (I) may be effected by a compound of the general formula (I) being in the same unit dose as a chemotherapeutic or other anti-cancer agent, or the compound of the general formula (I) and the chemotherapeutic or other anti-cancer agents may be present in individual and discrete unit doses administered at the same, or at a similar time. Sequential administration may be in any order as required, and may require an ongoing physiological effect of the first or initial compound to be current when the second or later compound is administered, especially where a cumulative or synergistic effect is desired.

Embodiments of the invention will now be discussed in more detail with reference to the examples which is provided for exemplification only and which should not be considered limiting on the scope of the invention in any way.

### EXAMPLES

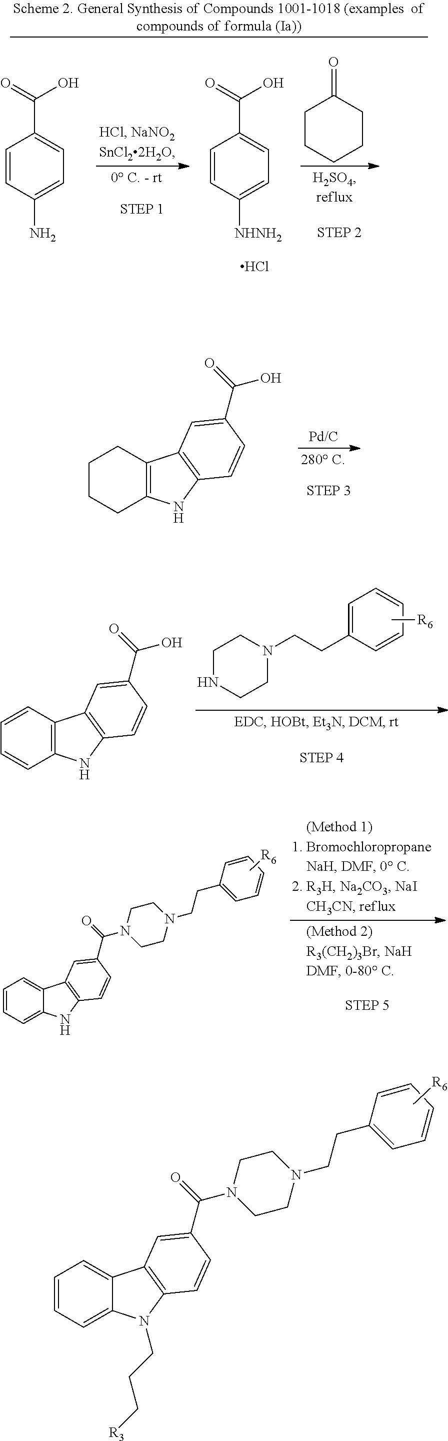

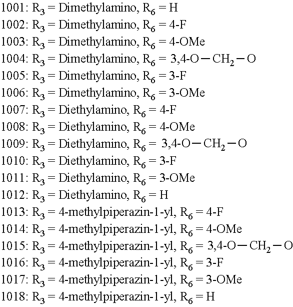

### Step 1: Preparation of 4-hydrazinylbenzoic acid hydrochloride

An aqueous solution of NaNO₂ (17.62 g, 255.5 mmol) was added to a stirred solution of 4-aminobenzoic acid (35.00 g, 255.5 mmol), in conc. HCl (105 mL) and ice-cold water (157 mL) at 0° C. The mixture was stirred for 30 minutes. A solution of SnCl₂.2H₂O (172.90 g, 766.41 mmol), in conc. HCl (173 mL) was added to the reaction mixture at 0° C. and the mixture was stirred for an additional 30 minutes. The obtained solids were filtered, washed with CH₃CN (2×100 mL), and dried under vacuum to afford 4-hydrazinylbenzoic acid hydrochloride as a milky white solid (35.0 g, 90%). LCMS: m/z 153.2 [(M-HCl)+H]⁺.

### Step 2: Preparation of 2,3,4,9-tetrahydro-1H-carbazole-6-carboxylic acid

A solution of 4-hydrazinylbenzoic acid hydrochloride (35.0 g, 186 mmol) in cyclohexanone (26.5 mL) was heated to 70° C. and stirred for 30 minutes. Conc. H₂SO₄ (42.0 mL) was added slowly to the reaction mixture, which was stirred at the same temperature for 6 hours. After complete consumption of the starting material, the reaction mixture was cooled to room temperature, neutralized with aqueous NaHCO₃ solution and extracted with ethyl acetate. The combined organic layers were dried over anhydrous Na₂SO₄ and concentrated under reduced pressure to obtain the crude product. Trituration with diethyl ether and n-pentane to afforded 2,3,4,9-tetrahydro-1H-carbazole-6-carboxylic acid as a brown solid (18 g, 45%). LCMS: m/z 216.07 [M+H]⁺.

### Step 3: Preparation of 9H-carbazole-3-carboxylic acid

2,3,4,9-Tetrahydro-1H-carbazole-6-carboxylic acid (2.50 g, 11.6 mmol) and 10% Pd/C (1.30 g, 50% w/w) were mixed together uniformly in a round bottom flask and then heated to 250° C. and stirred for 6 hours. The reaction was cooled to room temperature and stirred for 30 minutes. After the addition of methanol (50 mL) the mixture was filtered through Celite, which was washed with additional methanol (50 mL). The combined filtrates were concentrated under reduced pressure to give the crude product. The crude compound was purified by column chromatography on silica gel, eluting with 50% ethyl acetate in petroleum ether to afford 9H-carbazole-3-carboxylic acid (550 mg, 22%). LCMS: m/z 210.1 [M−H]⁻.

### Step 4: Preparation of (9H-carbazol-3-yl)(4-phenethylpiperazin-1-yl)methanone

To a stirred solution of 9H-carbazole-3-carboxylic acid (0.200 g, 0.946 mmol) in DMF (4 mL) was added HATU (0.720 g, 1.89 mmol) and DIPEA (0.50 mL, 2.8 mmol). The mixture was stirred for 30 minutes at room temperature. The reaction mixture was cooled to 0° C., 1-phenethylpiperazine (0.18 mL, 0.946 mmol) was added and the resulting reaction mixture was stirred for 16 hours at room temperature. After complete consumption of the starting material, ice-cold water was added to the reaction mixture, which was then extracted with ethyl acetate. The organic layers were dried over anhydrous Na₂SO₄ and concentrated under reduced pressure to afford the crude product. The crude compound was purified by flash column chromatography using 80% ethyl acetate in petroleum ether as an eluent, to afford (9H-carbazol-3-yl)(4-phenethylpiperazin-1-yl)methanone as a brown semi solid (300 mg, 33%). LCMS: m/z 384.12 [M+H]⁺.

Other analogues prepared by this method:

(9H-carbazol-3-yl)(4-(4-fluorophenethyl)piperazin-1-yl)methanone (78%). LCMS: m/z 402.23 [M+H]⁺.

(9H-carbazol-3-yl)(4-(4-methoxyphenethyl)piperazin-1-yl)methanone (34%). LCMS: m/z 414.18 [M+H]⁺.

(4-(2-(benzo[d][1,3]dioxol-5-yl)ethyl)piperazin-1-yl)(9H-carbazol-3-yl)methanone (63%). LCMS: m/z 428.47 [M+H]⁺.

(9H-carbazol-3-yl)(4-(3-fluorophenethyl)piperazin-1-yl)methanone (95%). LCMS: m/z 402.47 [M+H]⁺.

(9H-carbazol-3-yl)(4-(3-methoxyphenethyl)piperazin-1-yl)methanone (99%). LCMS: m/z 414.11 [M+H]⁺.

### Step 5: (Method 1) Preparation of Compound 1013, (4-(4-fluorophenethyl)piperazin-1-yl)(9-(3-(4-methylpiperazin-1-yl)propyl)-9H-carbazol-3-yl)methanone (Compound 13)

To a stirred solution of NaH (43.0 mg, 1.10 mmol) in DMF (4 mL) at 0° C. was added (9H-carbazol-3-yl)(4-(4-fluorophenethyl)piperazin-1-yl)methanone (220 mg, 0.548 mmol). After stirring for 10 minutes, bromochloropropane (0.30 mL, 3.0 mmol) was added at the same temperature and the reaction mass was slowly warmed to room temperature and stirred for 12 hours. After complete consumption of the starting material, ice-cold water was added to the reaction mixture, which was then extracted with ethyl acetate. The organic layers were dried over anhydrous Na₂SO₄ and concentrated under reduced pressure in order to afford the crude intermediate. The intermediate was purified by flash column chromatography eluted in 40% ethyl acetate in petroleum ether to afford (9-(3-chloropropyl)-9H-carbazol-3-yl)(4-(4-fluorophenethyl)piperazin-1-yl)methanone.

To the (9-(3-chloropropyl)-9H-carbazol-3-yl)(4-(4-fluorophenethyl)piperazin-1-yl)methanone was added N-methylpiperazine (0.10 ml, 0.90 mmol), Na₂CO₃ (110 mg, 1.04 mmol), NaI (156 mg, 1.04 mmol) and CH₃CN (4 mL). The mixture was heated at reflux for 12 hours. After complete consumption of the starting material, the solvent removed by distillation, ice-cold water was added and the mixture was extracted with ethyl acetate. The organic layers were dried over anhydrous Na₂SO₄ and concentrated under reduced pressure to afford the crude product. The crude compound was purified by preparative TLC using 5% MeOH in DCM as an eluent to afford (4-(4-fluorophenethyl)piperazin-1-yl)(9-(3-(4-methylpiperazin-1-yl)propyl)-9H-carbazol-3-yl)methanone (Compound 1013) as an off-white solid (22 mg, 10%).

¹H NMR (400 MHz, CD₃OD): δ 8.21 (d, J=1.2 Hz, 1H), 8.14 (d, J=7.6 Hz, 1H), 7.64 (d, J=8.4 Hz, 1H), 7.60 (d, J=8.4 Hz, 1H), 7.54 (dd, J=8.0 Hz, 1.2 Hz, 1H), 7.49 (t, J=7.2 Hz, 1H), 7.29-7.17 (m, 3H), 6.99 (t, J=8.8 Hz, 2H), 4.50 (t, J=6.4 Hz, 2H), 3.74 (br s, 4H), 2.82 (dd, J=10.4 Hz, 7.2 Hz, 2H), 2.68-2.34 (m, 14H), 2.33 (t, J=7.2 Hz, 2H), 2.08 (quintet, J=7.2 Hz, 2H). LCMS: m/z 542.46 [M+H]⁺.

Other analogues prepared by this method:

### Compound 1004, (4-(2-(benzo[d][1,3]dioxol-5-yl)ethyl)piperazin-1-yl)(9-(3-(dimethylamino)propyl)-9H-carbazol-3-yl)methanone (9%)

¹H NMR (300 MHz, CD₃OD): δ 8.23 (br s, 1H), 8.16 (d, J=8.1 Hz, 1H), 7.69-7.46 (m, 4H), 7.27 (t, J=7.5 Hz, 1H), 6.73-6.66 (m, 3H), 5.88 (s, 2H), 4.51 (t, J=6.9 Hz, 2H), 3.75 (br s, 4H), 2.81-2.53 (m, 10H), 2.43 (s, 6H), 2.22-2.14 (m, 2H). LCMS: m/z 513.45 [M+H]⁺.

### Compound 1014, (4-(4-methoxyphenethyl)piperazin-1-yl)(9-(3-(4-methylpiperazin-1-yl)propyl)-9H-carbazol-3-yl)methanone (14%)

¹H NMR (400 MHz, DMSO-d₆): δ 8.23-8.21 (m, 2H), 7.65 (d, J=8.4 Hz, 2H), 7.50-7.46 (m, 2H), 7.22 (t, J=7.6 Hz, 1H), 7.14 (d, J=8.4 Hz, 2H), 6.83 (d, J=8.4 Hz, 2H), 4.45 (t, J=6.8 Hz, 2H), 3.71 (s, 3H), 3.56 (br s, 4H), 2.71-2.66 (m, 2H), 2.54-2.50 (m, 6H), 2.33-2.18 (m, 10H), 2.15 (s, 3H), 1.96-1.92 (m, 2H). LCMS: m/z 554.0 [M+H]⁺.

### Compound 1015, (4-(2-(benzo[d][1,3]dioxol-5-yl)ethyl)piperazin-1-yl)(9-(3-(4-methylpiperazin-1-yl)propyl)-9H-carbazol-3-yl)methanone (5%)

¹H NMR (300 MHz, CD₃OD): δ 8.23 (d, J=1.5 Hz, 1H), 8.16 (d, J=7.5 Hz, 1H), 7.66 (d, J=8.4 Hz, 1H), 7.62 (d, J=8.4 Hz, 1H), 7.57-7.48 (m, 2H), 7.26 (t, J=7.5 Hz, 1H), 6.77-6.60 (m, 3H), 5.89 (s, 2H), 4.54 (t, J=6.3 Hz, 2H), 3.76 (br s, 4H), 2.92-2.36 (m, 21H), 2.13-2.18 (m, 2H). LCMS: m/z 568.49 [M+H]⁺.

### Compound 1016, (4-(3-fluorophenethyl)piperazin-1-yl)(9-(3-(4-methylpiperazin-1-yl)propyl)-9H-carbazol-3-yl)methanone (9%)

¹H NMR (400 MHz, CD₃OD): δ 8.21 (br s, 1H), 8.14 (d, J=8.0 Hz, 1H), 7.64 (d, J=8.4 Hz, 1H), 7.60 (d, J=8.4 Hz, 1H), 7.57-7.45 (m, 2H), 7.30-7.22 (m, 2H), 7.05 (br d, J=8.0 Hz, 1H), 6.99 (br d, J=10.0 Hz, 1H), 6.91 (br t, J=8.4 Hz, 1H), 4.50 (t, J=6.4 Hz, 2H), 3.74 (br s, 4H), 2.91-2.83 (m, 2H), 2.71-2.30 (m, 16H), 2.25 (s, 3H), 2.11-2.05 (m, 2H). LCMS: m/z 542.0 [M+H]⁺.

### Compound 1017, (4-(3-methoxyphenethyl)piperazin-1-yl)(9-(3-(4-methylpiperazin-1-yl)propyl)-9H-carbazol-3-yl)methanone (12%)

¹H NMR (300 MHz, CD₃OD): δ 8.22 (d, J=1.5 Hz, 1H), 8.14 (d, J=7.8 Hz, 1H), 7.70-7.43 (m, 4H), 7.23-7.17 (m, 2H), 6.84-6.72 (m, 3H), 4.50 (t, J=6.6 Hz, 2H), 3.88-3.61 (m, 7H), 2.86-2.79 (m, 2H), 2.70-2.29 (m, 16H), 2.27 (s, 3H), 2.11-2.05 (m, 2H). LCMS: m/z 554.26 [M+H]⁺.

### Compound 1018, (4-phenethylpiperazin-1-yl)(9-(3-(4-methylpiperazin-1-yl)propyl)-9H-carbazol-3-yl)methanone (20%)

¹H NMR (400 MHz, CD₃OD): δ 8.22 (br s, 1H), 8.14 (d, J=7.6 Hz, 1H), 7.67-7.42 (m, 4H), 7.30-7.14 (m, 6H), 4.50 (t, J=6.4 Hz, 2H), 3.75 (br s, 4H), 2.87-2.80 (m, 2H), 2.73-2.40 (m, 19H), 2.07 (quintet, J=6.8 Hz, 2H). LCMS: m/z 524.20 [M+H]⁺.

### Step 5: (Method 2) Preparation of Compound 1002, (9-(3-(dimethylamino)propyl)-9H-carbazol-3-yl)(4-(4-fluorophenethyl)piperazin-1-yl)methanone

To a stirred solution of NaH (134 mg, 3.34 mmol) in DMF (4 mL) at 0° C. was added (9H-carbazol-3-yl)(4-(4-fluorophenethyl)piperazin-1-yl)methanone (0.168 g, 0.418 mmol). The mixture was stirred for 10 minutes, after which N(CH₃)₂CH₂CH₂Br (0.328 g, 2.091 mmol) was added at the same temperature. The reaction mass was slowly warmed to room temperature and stirred for 30 minutes, then heated to 80° C. for 12 hours. After complete consumption of the starting material, ice-cold water was added and the reaction mixture was extracted with ethyl acetate. The organic layers were dried over anhydrous Na₂SO₄ and concentrated under reduced pressure to afford the crude product. The crude product was purified by preparative TLC eluted in 5% methanol in CH₂Cl₂, to give (9-(3-(dimethylamino)propyl)-9H-carbazol-3-yl)(4-(4-fluorophenethyl)piperazin-1-yl)methanone (Compound 1002) as a yellow solid (30 mg, 13%).

¹H NMR (400 MHz, CD₃OD): δ 8.22 (br s, 1H), 8.15 (d, J=7.8 Hz, 1H), 7.67-7.46 (m, 4H), 7.33-7.16 (m, 3H), 6.99 (t, J=8.7 Hz, 2H), 4.48 (t, J=6.9 Hz, 2H), 3.74 (br s, 4H), 2.89-2.73 (m, 2H), 2.75-2.55 (m, 6H), 2.44-2.31 (m, 2H), 2.20 (s, 6H), 2.13-1.99 (m, 2H). LCMS: m/z 487.0 [M+H]⁺.

Other analogues prepared by this method:

### Compound 1001, (9-(3-(dimethylamino)propyl)-9H-carbazol-3-yl)(4-phenethylpiperazin-1-yl)methanone (10%)

¹H NMR (300 MHz, CD₃OD): δ 8.22 (d, J=1.5 Hz, 1H), 8.15 (d, J=7.5 Hz, 1H), 7.64-7.47 (m, 4H), 7.30-7.13 (m, 6H), 4.48 (t, J=6.9 Hz, 2H), 3.75 (br s, 4H), 2.87-2.80 (m, 2H), 2.71-2.56 (m, 6H), 2.42 (dd, J=9.3 Hz, 6.9 Hz, 2H), 2.25 (s, 6H), 2.08 (quintet, J=7.5 Hz, 2H). LCMS: m/z 469.16 [M+H]⁺.

### Compound 1003, (9-(3-(dimethylamino)propyl)-9H-carbazol-3-yl)(4-(4-methoxyphenethyl)piperazin-1-yl)methanone (14%)

¹H NMR (300 MHz, CD₃OD): δ 8.22 (d, J=1.2 Hz, 1H), 8.15 (d, J=7.8 Hz, 1H), 7.67-7.45 (m, 4H), 7.26 (t, J=6.9 Hz, 1H), 7.13 (d, J=8.4 Hz, 2H), 6.83 (d, J=8.7 Hz, 2H), 4.49 (t, J=7.2 Hz, 2H), 3.91-3.52 (m, 7H), 2.81-2.76 (m, 2H), 2.67-2.58 (m, 6H), 2.51-2.38 (m, 2H), 2.28 (s, 6H), 2.10 (quintet, J=7.2 Hz, 2H). LCMS: m/z 499.49 [M+H]⁺.

### Compound 1005, (9-(3-(dimethylamino)propyl)-9H-carbazol-3-yl)(4-(3-fluorophenethyl)piperazin-1-yl)methanone (13%)

¹H NMR (300 MHz, CD₃OD): δ 8.22 (d, J=1.2 Hz, 1H), 8.15 (d, J=7.5 Hz, 1H), 7.66-7.46 (m, 4H), 7.30-7.21 (m, 2H), 7.05 (br d, J=7.5 Hz, 1H), 6.99 (br d, J=10.2 Hz, 1H), 6.91 (td, J=8.7 Hz, 2.1 Hz, 1H), 4.50 (t, J=6.9 Hz, 2H), 3.75 (br s, 4H), 2.88-2.83 (m, 2H), 2.73-2.53 (m, 8H), 2.37 (s, 6H), 2.16-2.11 (m, 2H). LCMS: m/z 487.48 [M+H]⁺.

### Compound 1006, (9-(3-(dimethylamino)propyl)-9H-carbazol-3-yl)(4-(3-methoxyphenethyl)piperazin-1-yl)methanone (9%)

¹H NMR (300 MHz, DMSO-d₆): δ 8.28-8.22 (m, 2H), 7.66 (d, J=8.7 Hz, 2H), 7.53-7.46 (m, 2H), 7.24 (t, J=7.8 Hz, 1H), 7.19 (t, J=8.1 Hz, 1H), 6.84-6.70 (m, 3H), 4.46 (t, J=6.3 Hz, 2H), 3.73 (s, 3H), 3.57 (br s, 4H), 2.73-2.44 (m, 10H), 2.43-2.21 (s, 6H), 2.01-1.91 (m, 2H). LCMS: m/z 499.48 [M+H]⁺.

### Compound 1007, (9-(3-(diethylamino)propyl)-9H-carbazol-3-yl)(4-(4-fluorophenethyl)piperazin-1-yl)methanone (12%)

¹H NMR (400 MHz, CD₃OD): δ 8.22 (d, J=1.2 Hz, 1H), 8.14 (d, J=7.6 Hz, 1H), 7.62 (d, J=8.4 Hz, 1H), 7.58 (d, J=8.4 Hz, 1H), 7.54 (dd, J=8.8 Hz, 1.6 Hz, 1H), 7.50 (td, J=8.0 Hz, 0.8 Hz, 1H), 7.29-7.18 (m, 3H), 6.99 (t, J=8.8 Hz, 2H), 4.47 (t, J=6.8 Hz, 2H), 3.74 (br s, 4H), 2.84-2.80 (dd, J=10.8 Hz, 7.6 Hz, 2H), 2.68-2.55 (m, 6H), 2.54-2.42 (m, 6H), 2.10-1.98 (m, 2H), 0.94 (t, J=7.2 Hz, 6H). LCMS: m/z 515.13 [M+H]⁺.

### Compound 1008, (9-(3-(diethylamino)propyl)-9H-carbazol-3-yl)(4-(4-methoxyphenethyl)piperazin-1-yl)methanone (11%)

¹H NMR (400 MHz, CD₃OD): δ 8.22 (d, J=1.2 Hz, 1H), 8.15 (d, J=8.0 Hz, 1H), 7.62 (d, J=8.4 Hz, 1H), 7.58 (d, J=8.4 Hz, 1H), 7.55 (dd, J=8.4 Hz, 1.6 Hz, 1H), 7.50 (t, J=7.2 Hz, 1H), 7.25 (t, J=7.2 Hz, 1H), 7.13 (d, J=8.4 Hz, 2H), 6.83 (d, J=8.8 Hz, 2H), 4.47 (t, J=6.8 Hz, 2H), 3.88-3.59 (m, 7H), 2.79-2.75 (m, 2H), 2.65-2.58 (m, 6H), 2.57-2.44 (m, 6H), 2.12-2.00 (m, 2H). 0.95 (t, J=7.2 Hz, 6H). LCMS: m/z 527.48 [M+H]⁺.

### Compound 1009, (4-(2-(benzo[d][1,3]dioxol-5-yl)ethyl)piperazin-1-yl)(9-(3-(diethylamino)propyl)-9H-carbazol-3-yl)methanone (6%)

¹H NMR (300 MHz, CD₃OD): δ 8.23 (br s, 1H), 8.16 (d, J=8.1 Hz, 1H), 7.60-7.46 (m, 4H), 7.26 (t, J=7.2 Hz, 1H), 6.74-6.66 (m, 3H), 5.88 (s, 2H), 4.50 (t, J=6.9 Hz, 2H), 3.74 (br s, 4H), 2.81-2.68 (m, 14H), 2.12-2.07 (m, 2H), 1.00 (t, J=7.2 Hz, 6H). LCMS: m/z 541.50 [M+H]⁺.

### Compound 1010, (9-(3-(diethylamino)propyl)-9H-carbazol-3-yl)(4-(3-fluorophenethyl)piperazin-1-yl)methanone (8%)

¹H NMR (400 MHz, CD₃OD): δ 8.22 (br s, 1H), 8.16 (d, J=8.0 Hz, 1H), 7.63 (d, J=8.4 Hz, 1H), 7.59 (d, J=8.0 Hz, 1H), 7.55 (dd, J=8.4 Hz, 1.6 Hz, 1H), 7.50 (t, J=7.6 Hz, 1H), 7.28-7.23 (m, 2H), 7.05 (br d, J=7.6 Hz, 1H), 6.99 (br d, J=10.4 Hz, 1H), 6.94-6.88 (m, 1H), 4.47 (t, J=7.2 Hz, 2H), 3.74 (br s, 4H), 2.88-2.84 (m, 2H), 2.70-2.67 (m, 6H), 2.52-2.48 (m, 6H), 2.15-2.05 (m, 2H), 0.94 (t, J=7.2 Hz, 6H). LCMS: m/z 515.17 [M+H]⁺.

### Compound 1011, (9-(3-(diethylamino)propyl)-9H-carbazol-3-yl)(4-(3-methoxyphenethyl)piperazin-1-yl)methanone (9%)

¹H NMR (300 MHz, DMSO-d₆): δ 8.27-8.22 (m, 2H), 7.73-7.64 (m, 2H), 7.54-7.48 (m, 2H), 7.28-7.14 (m, 2H), 6.83-6.72 (m, 3H), 4.52-4.46 (m, 2H), 3.73 (s, 3H), 3.39 (br s, 4H), 2.76-2.68 (m, 2H), 2.57-2.38 (m, 12H), 2.02-1.98 (m, 2H), 1.11 (t, J=7.2 Hz, 6H). LCMS: m/z 527.48 [M+H]⁺.

### Compound 1012, (9-(3-(diethylamino)propyl)-9H-carbazol-3-yl)(4-phenethylpiperazin-1-yl)methanone (18%)

¹H NMR (400 MHz, CD₃OD): δ 8.23 (br s, 1H), 8.16 (d, J=7.6 Hz, 1H), 7.64 (d, J=8.4 Hz, 1H), 7.60 (d, J=8.4 Hz, 1H), 7.56 (dd, J=8.4 Hz, 1.6 Hz, 1H), 7.52 (t, J=7.2 Hz, 1H), 7.29-7.20 (m, 5H), 7.17 (t, J=7.2 Hz, 1H), 4.52 (t, J=6.8 Hz, 2H), 3.76 (br s, 4H), 2.89-2.70 (m, 8H), 2.72-2.52 (m, 6H), 2.19-2.13 (m, 2H), 1.03 (t, J=7.2 Hz, 6H). LCMS: m/z 497.17 [M+H]⁺.

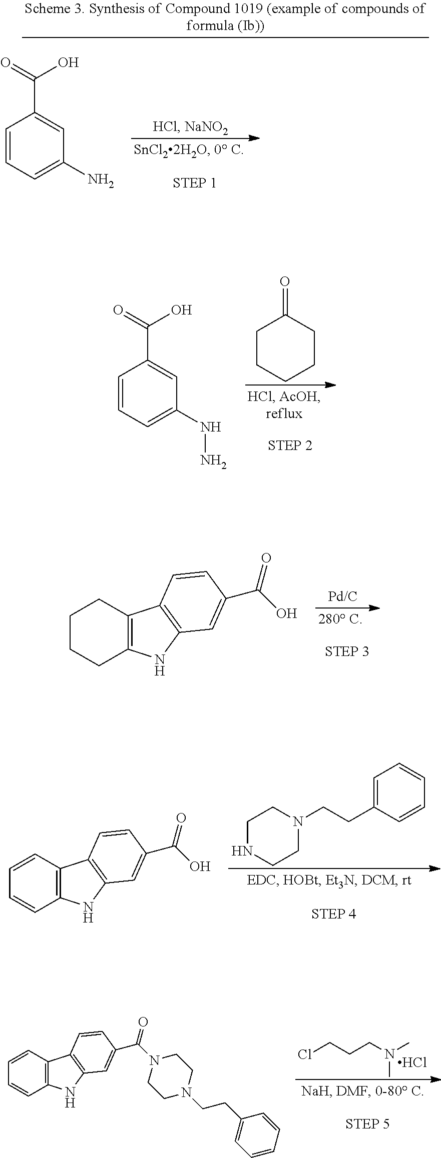

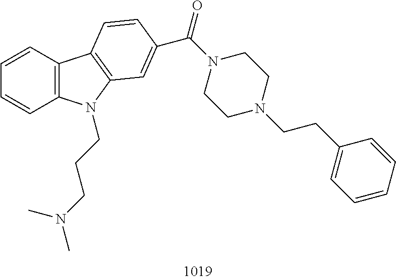 [SMILES: CN(C)CCCn1c2ccccc2c2ccc(C(=O)N3CCN(CCc4ccccc4)CC3)cc21]

### Step 1: Preparation of 3-hydrazinylbenzoic acid

To a stirred suspension of 3-aminobenzoic acid (2 g, 14.6 mmol) in conc. HCl was added an aqueous solution of NaNO₂ (1 g, 14.6 mmol) at 0° C. The reaction mixture was stirred for 1 hour. A solution of SnCl₂.2H₂O in conc. HCl was then added at 0° C. The reaction solution was stirred for an additional 2 hours at room temperature. The precipitate was filtered and washed with ethanol and ether to give the crude product, which was purified by column chromatography on silica gel to give 3-hydrazinylbenzoic acid as a pale solid (1.8 g, 81%).

### Step 2: Preparation of 2,3,4,9-tetrahydro-1H-carbazole-7-carboxylic acid

To a stirred suspension of 3-hydrazinylbenzoic acid (200 mg, 1.32 mmol) in acetic acid was added cyclohexanone (129 mg, 1.32 mmol) and conc. HCl. The mixture was heated at reflux for 3 hours and cooled to room temperature. The resultant was concentrated directly in vacuum to remove acetic acid, the residue was washed with water and extracted with ethyl acetate, and the organic layer was extracted with 2 N NaOH solution. The alkaline layers were made acidic by the addition of 2 N HCl, extracted with diethyl ether, dried and concentrated to give a crude product, which was purified by column chromatography on silica gel to give 2,3,4,9-tetrahydro-1H-carbazole-7-carboxylic acid as a pale solid (110 mg, 39%).

¹H NMR (400 MHz, DMSO-d₆): δ 12.36 (s, 1H), 11.04 (s, 1H), 7.88 (s, 1H), 7.55 (d, J=9.2 Hz, 1H), 7.38 (d, J=8.4 Hz, 1H), 2.73 (t, J=4.8 Hz, 2H), 2.64 (t, J=4.8 Hz, 2H), 1.84-1.81 (m, 4H).

### Step 3: Preparation of 9H-carbazole-2-carboxylic acid

2,3,4,9-Tetrahydro-1H-carbazole-7-carboxylic acid (330 mg, 1.53 mmol) and Pd/C (150 mg) were uniformly mixed and dropped into a flask. The mixture was heated to 280° C. for 3 hours. The resultant was cooled to room temperature and methanol was added. The mixture was filtered to remove Pd/C, concentrated and purified by column chromatography on silica gel to give 9H-carbazole-2-carboxylic acid as a pale solid (30 mg, 10%).

¹H NMR (400MHz, DMSO-d₆): δ 12.84 (s, 1H), 11.52 (s, 1H), 8.19 (d, J=5.6 Hz, 2H), 8.08 (s, 1H), 7.76 (d, J=6.8 Hz, 1H), 7.54 (d, J=10.0 Hz, 1H), 7.46 (t, J=7.2 Hz, 1H), 7.20 (t, J=6.8 Hz, 1H).

### Step 4: Preparation of (9H-carbazol-2-yl)(4-phenethylpiperazin-1-yl)methanone

To a stirred suspension of 1-phenethylpiperazine hydrochloride (50 mg, 0.22 mmol) in DCM was added Et₃N (29 mg, 0.28 mmol) and the mixture was stirred at room temperature for 30 minutes. 9H-Carbazole-2-carboxylic acid (30 mg, 0.14 mmol), EDC (54 mg, 0.28 mmol) and HOBt (38 mg, 0.28 mmol) were then added one by one at 0° C. The mixture was then allowed to warm to room temperature and stirred overnight. The resultant was quenched by water, extracted with ethyl acetate, washed with solutions of Na₂CO₃ and NH₄Cl, and then concentrated to give (9H-carbazol-2-yl)(4-phenethylpiperazin-1-yl)methanone as a colourless oil (70 mg, quant.).

### Step 5: Preparation of Compound 1019, (9-(3-(dimethylamino)propyl)-9H-carbazol-2-yl)(4-phenethylpiperazin-1-yl) methanone

To a stirred suspension of (9H-carbazol-2-yl)(4-phenethylpiperazin-1-yl)methanone (70 mg, 0.18 mmol) in DMF was added NaH (18 mg, 0.45 mmol) at 0° C. The mixture was then stirred at the same temperature for 30 minutes. 3-Chloro-N,N-dimethylpropan-1-amine hydrochloride (32 mg, 0.20 mmol) was added at 0° C. The reaction mixture was allowed to heat to 80° C. and stirred for 2 hours. It was then quenched with water, extracted with ethyl acetate, concentrated and purified by silica gel plates to give (9-(3-(dimethylamino)propyl)-9H-carbazol-2-yl)(4-phenethylpiperazin-1-yl) methanone (Compound 1019) as a colourless oil (15 mg, 18%).

¹H NMR (400 MHz, CDCl₃): δ 8.10 (t, J=7.6 Hz, 2H), 7.59 (s, 1H), 7.50 (d, J=4.0 Hz, 2H), 7.32-7.20 (m, 7H), 4.41 (t, J=6.8 Hz, 2H), 3.89-3.56 (m, 4H), 2.85-2.81 (m, 2H), 2.68-2.50 (m, 6H), 2.28 (t, J=6.4 Hz, 2H), 2.23 (s, 6H), 2.05-2.01 (m, 2H).

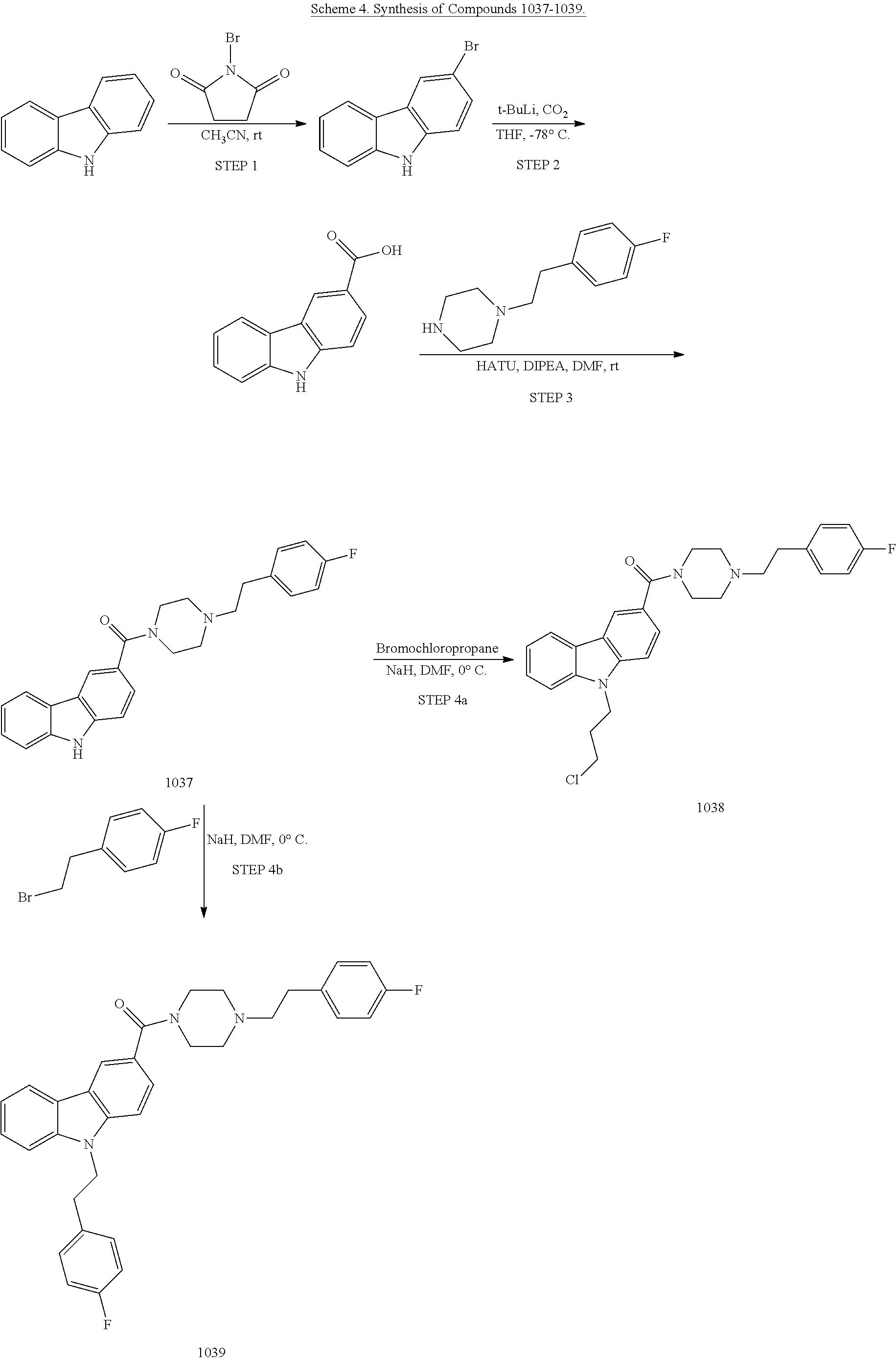

### Step 1: Preparation of 3-bromo-9H-carbazole

N-Bromosuccinimide (21.63 g, 121.53 mmol) was added portionwise to a stirred solution of 9H-carbazole (19.00 g, 113.6 mmol) in acetonitrile (420 mL) at room temperature. The reaction mixture was stirred at room temperature for 16 hours. After complete consumption of the starting material, as indicated by TLC, the reaction mixture was precipitated and filtered. The precipitate was washed with n-pentane to afford 3-bromo-9H-carbazole as an off-white solid (14.7 g, 53%).

¹H NMR (400 MHz, CDCl₃): δ 8.19 (br s, 1H), 8.05-7.97 (m, 2H), 7.54-7.40 (m, 3H), 7.32-7.25 (m, 2H). LCMS: m/z 244.03, 246.03 [M−H]⁻.

### Step 2: Preparation of 9H-carbazole-3-carboxylic acid

t-Butyllithium (25.0 mL, 36.57 mmol) was added slowly to dry THF (30 mL), at −78° C. A solution of 3-bromo-9H-carbazole (3.00 g, 12.19 mmol) in THF (30 mL) was added over 30 minutes with the temperature maintained at −78° C. The reaction mixture was maintained at this temperature for one hour while being slowly purged with dry CO₂ gas. After complete consumption of the starting material, as indicated by TLC, the reaction mixture was quenched with saturated ammonium chloride solution and 1 N HCl. The reaction mixture was warmed to room temperature and extracted with ethyl acetate. The organic layer was dried over anhydrous Na₂SO₄, filtered and concentrated under reduced pressure to get 9H-carbazole-3-carboxylic acid as an off-white solid (1.6 g, 62%). LCMS: m/z 210.1 [M−H]⁻.

### Step 3: Preparation of Compound 1037, (9H-carbazol-3-yl)(4-(4-fluorophenethyl)piperazin-1-yl)methanone

HATU (4.30 g, 11.36 mmol) was added to a stirred solution of 9H-carbazole-3-carboxylic acid (1.60 g, 7.57 mmol) and DIPEA (6.5 mL, 37.8 mmol) in DMF (20 mL) at room temperature. The reaction mixture was cooled to 0° C. prior to the addition of 1-(4-fluorophenethyl)piperazine (2.70 g, 11.35 mmol), then slowly allowed to warm to room temperature and stirred for 12 hours. After complete consumption of the starting material based on TLC, ice water was poured into the reaction mixture, which was then extracted with ethyl acetate. The organic layer was washed with water, followed by brine solution, then dried over anhydrous Na₂SO₄ and concentrated under reduced pressure to afford the crude product. The crude compound was purified by using 100-200 mesh silica gel, eluting with 40% ethyl acetate in petroleum ether to afford pure (9H-carbazol-3-yl)(4-(4-fluorophenethyl)piperazin-1-yl)methanone (Compound 1037) as a brown solid (1.85 g, 61%).

¹H NMR (400 MHz, CD₃OD): δ 8.19 (br s, 1H), 8.10 (d, J=7.8 Hz, 1H), 7.54-7.37 (m, 4H), 7.28-7.17 (m, 3H), 7.00 (t, J=8.7 Hz, 2H), 3.76 (br s, 4H), 2.87-2.81 (m, 2H), 2.71-2.59 (m, 6H). LCMS: m/z 402.62 [M+H]⁺.

### Step 4a: Preparation of Compound 1038, (9-(3-chloropropyl)-9H-carbazol-3-yl)(4-(4-fluorophenethyl)piperazin-1-yl)methanone

NaH (258 mg, 6.46 mmol) was added to a stirred solution of Compound 1037 (1.30 g, 3.23 mmol) in DMF (25 mL) at room temperature. The reaction mixture was cooled to 0° C. and stirred for 30 minutes. At this temperature was added bromochloropropane (0.79 mL, 8.07 mmol). The reaction mixture was stirred at 0° C. for two hours. After complete consumption of the starting material was indicated by TLC, the reaction mixture was quenched with ice cold water and extracted with ethyl acetate. The organic layer was dried over anhydrous Na₂SO₄ and concentrated under reduced pressure to afford the crude product. The crude compound was purified by using 100-200 mesh silica gel, eluting with 30% ethyl acetate in petroleum ether to afford pure (9-(3-chloropropyl)-9H-carbazol-3-yl)(4-(4-fluorophenethyl)piperazin-1-yl)methanone (Compound 1038) as an off-white sticky liquid (850 mg, 55%).

¹H NMR (300 MHz, DMSO-d₆): δ 8.27-8.22 (m, 2H), 7.66 (d, J=8.4 Hz, 2H), 7.54-7.47 (m, 2H), 7.30-7.24 (m, 3H), 7.09 (t, J=9.0 Hz, 2H), 4.55 (t, J=6.9 Hz, 2H), 3.66 (t, J=6.6 Hz, 2H), 3.57 (br s, 4H), 2.77-2.71 (m, 2H), 2.58-2.43 (m, 6H), 2.28-2.20 (m, 2H). LCMS: m/z 478.60 [M+H]⁺.

### Step 4b: Preparation of Compound 1039, (9-(4-fluorophenethyl)-9H-carbazol-3-yl)(4-(4-fluorophenethyl)piperazin-1-yl)methanone

NaH (30 mg, 0.74 mmol) was added to a stirred solution of Compound 1037 (0.15 g, 0.37 mmol) in DMF (5 mL) at room temperature. The reaction mixture was cooled to 0° C. and stirred for 30 minutes. 1-(2-bromoethyl)-4-fluorobenzene (120 mg, 0.59 mmol) was added at 0° C. and this temperature was maintained for two hours. Then, the reaction mixture was slowly warmed to room temperature and stirred for 12 hours. After complete consumption of the starting material, based on TLC, ice water was added very slowly to the reaction mixture, which was then extracted with ethyl acetate. The organic layer was washed with water followed by brine solution, dried over anhydrous Na₂SO₄ and concentrated under reduced pressure to afford the crude product. The crude compound was purified by preparative TLC, to afford pure (9-(4-fluorophenethyl)-9H-carbazol-3-yl)(4-(4-fluorophenethyl)piperazin-1-yl)methanone (Compound 1039) as an off-white semisolid (40 mg, 61%).

¹H NMR (400 MHz, DMSO-d₆): δ 8.23-8.20 (m, 2H), 7.59 (d, J=8.4 Hz, 1H), 7.55 (d, J=8.8 Hz, 1H), 7.47-7.39 (m, 2H), 7.30-7.16 (m, 5H), 7.09 (t, J=8.8 Hz, 2H), 7.01 (t, J=8.8 Hz, 2H), 4.62 (t, J=7.2 Hz, 2H), 3.55 (br s, 4H), 3.05 (t, J=7.6 Hz, 2H), 2.77-2.73 (m, 2H), 2.58-2.42 (m, 6H). LCMS: m/z 524.46 [M+H]⁺.

Activity of Anti-Tropomyosin Compounds as Monotherapy

Anti-Proliferative Activity of Compounds of the Invention

In silico modelling has identified binding sites on tropomyosin Tpm3.1, yielding the series of tropomyosin inhibitors the subject of the present invention. Inhibition of Tpm3.1 in tumour cells results in disruption of the actin cytoskeleton and ultimately cell death.

The ability of compounds 1001-1018 and 1037-1039 to inhibit the proliferation of cancer cells representative of neuroblastoma, melanoma, prostate cancer, colorectal cancer, non-small cell lung carcinoma, and triple negative breast cancer was assessed (Table 1).

Briefly, a pre-determined number of cells as calculated from cell growth assays for each of the cell lines employed were seeded into their respective culture medium (using ATCC culture parameters—http://www.atcc.org) and cultured for 24 hours at 37° C. and 5% CO₂ in 96-well culture plates. Once attached, each cell line was then exposed to increasing concentrations of each respective analogue (0.03, 0.3, 3 and 30 μM for compounds 1001-13 and 1015-18; 0.1, 0.3, 1, 3, 10 and 30 μM for compounds 1014 and 1037-39), cultured for a further 72 hours and exposed to cell-titre luminescent reagent (100 μL/well) for a further 30 minutes) to measure cell viability. Luminescence was captured using an EnVision multilabel reader and the data for each analogue concentration compared against no treatment control. For compounds 1001-13 and 1015-18, semi-log plots of Percent of Control versus concentration were prepared and IC₅₀ determined using linear regression analysis. For compounds 1014 and 1037-39, cell viability was normalized to control (vehicle alone) and dose-response curves, and half maximal effective concentration (EC₅₀) values were determined using Graph Pad Prism 6 (nonlinear regression sigmoidal dose-response variable slope).

TABLE 1
Anti-proliferative activity of compounds of the invention against a range of
somatic cancer cells.

| | IC₅₀/μM | | | | | | |
|---|---|---|---|---|---|---|---|
| | Neuro | | | | | Lung | |
| Compound | blastoma | Melanoma | Prostate | | Colorectal | (NSLC) | Breast |
| ID | SK-N-SH | SK-Mel-28 | DU145 | PC3 | CaCo2 | A549 | MDA-MB-231 |
| 1001 | — | 2.3 | >30 | 5.5 | — | 8.5 | 4.4 |
| 1002 | 3.4 | 3.7 | 3.7 | 6.1 | 3.7 | 3.4 | 4.5 |
| 1003 | 3.1 | 3.2 | 3.6 | 3.1 | 3.7 | 3.4 | 4.3 |
| 1004 | 2.9 | 3.5 | 3.3 | 2.9 | 3.7 | 3.4 | 3.8 |
| 1005 | 3.3 | 3.5 | 3.2 | 3.3 | 3.8 | 3.5 | 4.1 |
| 1006 | 3.4 | 3.5 | 4.4 | 5.2 | 4.1 | 3.5 | 3.0 |
| 1007 | 1.6 | 1.2 | 3.0 | 3.2 | 2.8 | 1.9 | 2.4 |
| 1008 | 3.3 | 2.5 | 4.0 | 4.8 | 6.7 | 3.8 | 3.5 |
| 1009 | 4.8 | 4.1 | 12.1 | 4.6 | 5.0 | 4.0 | 4.5 |
| 1010 | 3.3 | 3.9 | 3.7 | 3.7 | 4.1 | 3.6 | 4.2 |
| 1011 | 4.2 | 3.4 | 4.5 | 5.5 | 4.0 | 3.8 | 2.5 |
| 1012 | 2.9 | 2.8 | 3.5 | 4.1 | 2.8 | 3.2 | 3.1 |
| 1013 | 2.4 | 2.0 | 3.5 | 3.3 | 2.0 | 3.7 | 2.8 |
| 1014 | 8.8 | 5.1 | 40.2 | 3.8 | 13.8 | 31.1 | 10.2 |
| 1015 | 5.1 | 3.4 | 15.5 | 5.3 | 5.5 | 4.0 | 4.4 |
| 1016 | 2.7 | 2.4 | 2.8 | 4.0 | 3.1 | 3.0 | 2.5 |
| 1017 | 4.7 | 4.0 | 6.6 | 5.2 | 4.6 | 3.6 | 3.7 |
| 1018 | 1.9 | 2.5 | 3.3 | 2.9 | 1.5 | 2.0 | 1.6 |
| 1037 | >30 | >30 | >30 | >30 | >30 | 16.34 | >30 |
| 1038 | >30 | >30 | >30 | >30 | >30 | >30 | >30 |
| 1039 | >30 | >30 | >30 | >30 | >30 | >30 | >30 |

Impact of Compounds of the Invention on the Actin Cytoskeleton

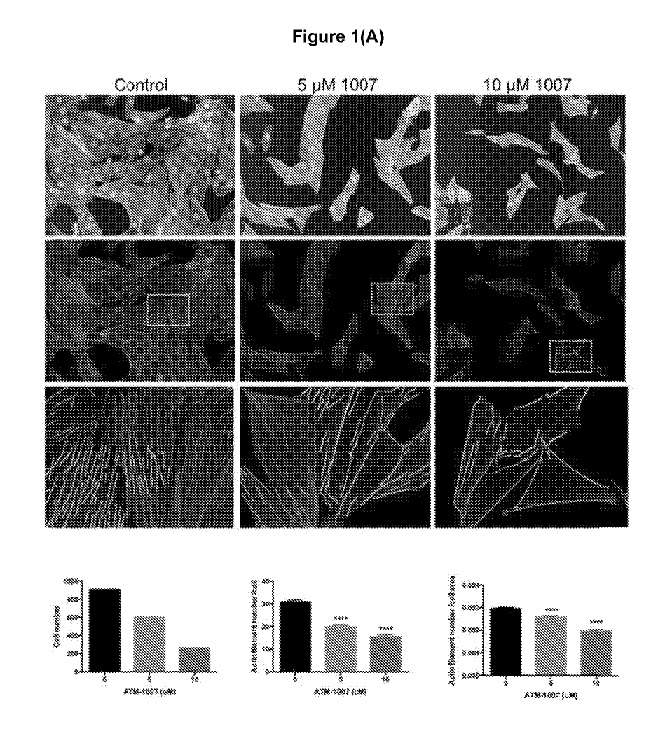

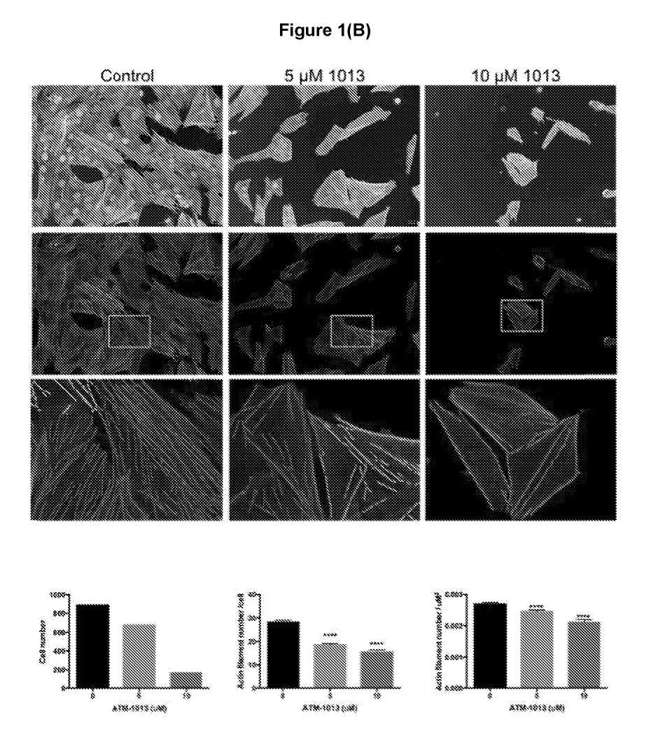

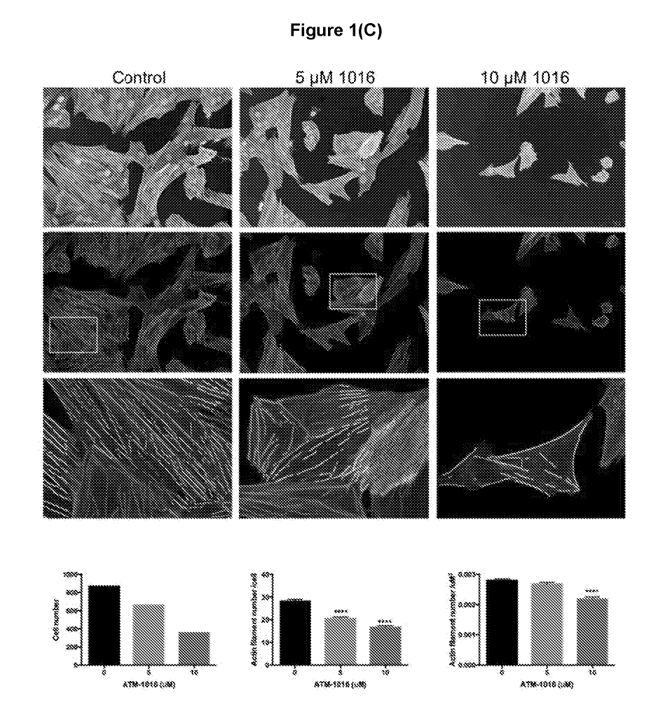

The ability of compounds 1007, 1013 and 1016 to disrupt the actin cytoskeleton was assessed in vitro using the microfilament disruption assay (FIG. 1).

Briefly, SK-N-SH neuroblastoma cells were seeded at 1800 cells/well in a 384 Perkin Elmer High Content Imaging “Cell Carrier” plate and left to plate down 24 hours prior to treatment. Cells were then treated with 0-40 μM of the test compounds (1:2 serial dilution in a 10 point dose response). 24 hours post treatment, cells were fixed with 4% w/v paraformaldehyde (PBS), permeabilized with Triton-X-100 and stained with 488-Atto-Phallodin and DAPI to visualize the actin filament bundles and the nucleus respectively. Single plane images were obtained on the Perkin Elmer Opera confocal microscope using a 20× objective. Twelve fields of view per condition (representing approximately 300-800 cells) were imaged. Images were then exported and changes in the organization and numbers of actin filaments within the cell were quantitated using a linear feature detection algorithm developed by CSIRO (Vindin et al. 2014). This algorithm detects the “ridge lines” or “peaks” in local pixel intensity in the cell image. It is these “ridge lines” that correspond to actin filament bundles and allow us to quantitate the number of filaments per cell.

Data demonstrate that compounds 1007, 1013 and 1016 disrupt the actin cytoskeleton in a dose-dependent manner.

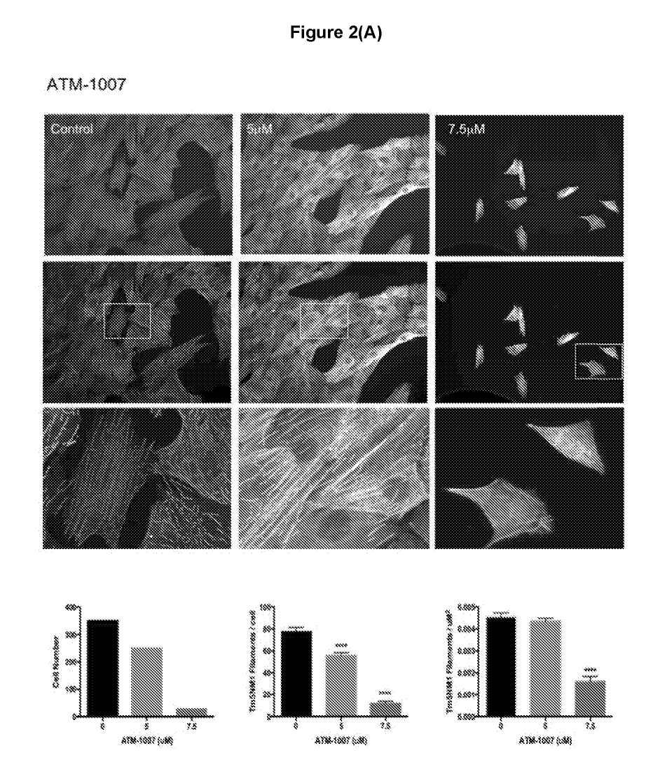

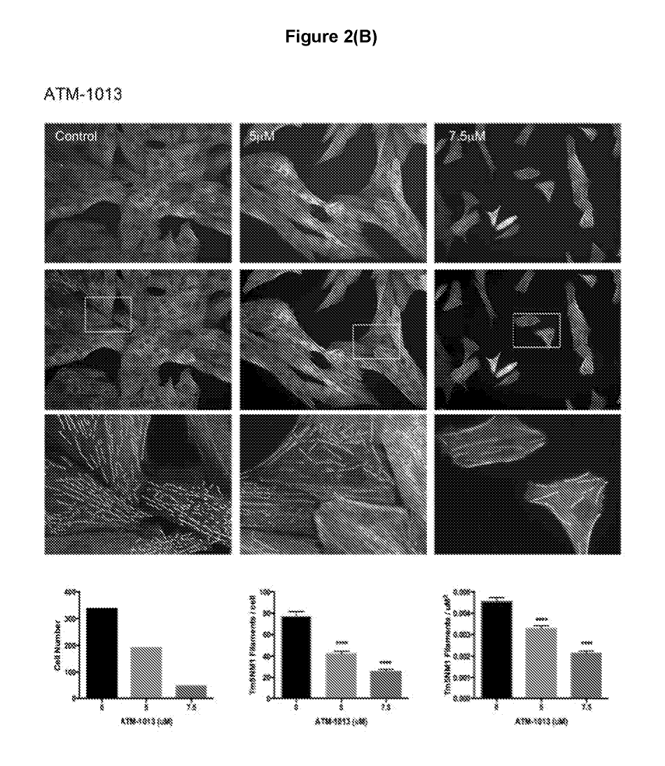

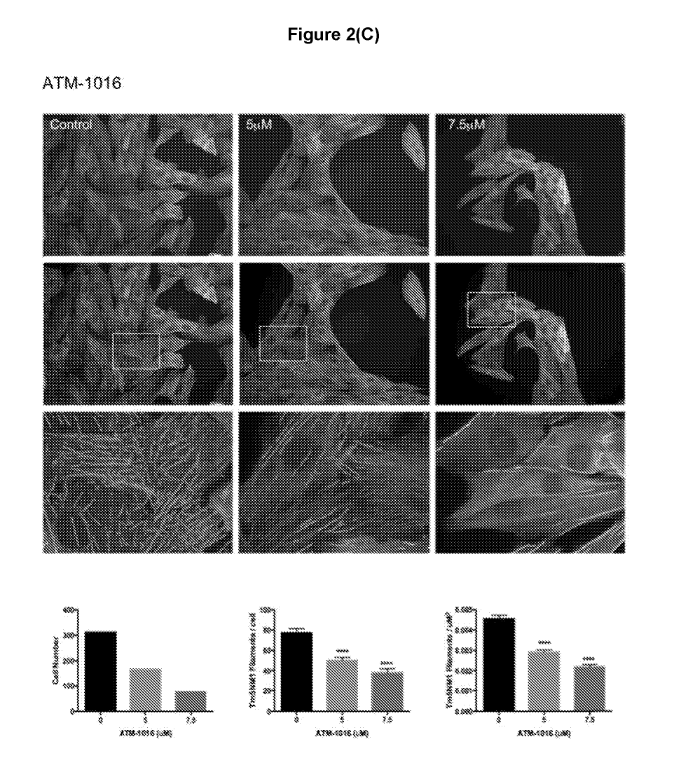

Another assay was conducted to assess the ability of compounds 1007, 1013 and 1016 to specifically disrupt actin filaments containing Tpm3.1 (FIG. 2). Briefly, SK-N-SH neuroblastoma cells were seeded at 2×10⁴ cells/mL in a 10 cm tissue culture plate and left to plate down 24 hours prior to treatment. Cells were then treated with 0, 5 and 7.5 μM of the test compounds. 48 hours post treatment, cells were fixed with 4% w/v paraformaldehyde (PBS), permeabilized with Triton-X-100 and stained with γ9d (sheep polycolonal, 1:100) followed by 488-conjugated secondary (1:1000) and DAPI to visualize the Tpm3.1 containing filament bundles and the nucleus, respectively. Single plane images were obtained on the Zeiss epifluorescent Axioscope microscope using a 20× objective. Six fields of view per condition were imaged. Images were then exported and the linear features (actin filaments containing Tpm3.1) were then quantitated using the linear feature detection algorithm described above.

Data demonstrate that compounds 1007, 1013 and 1016 disrupt Tpm3.1-containing actin filaments in a dose-dependent manner.

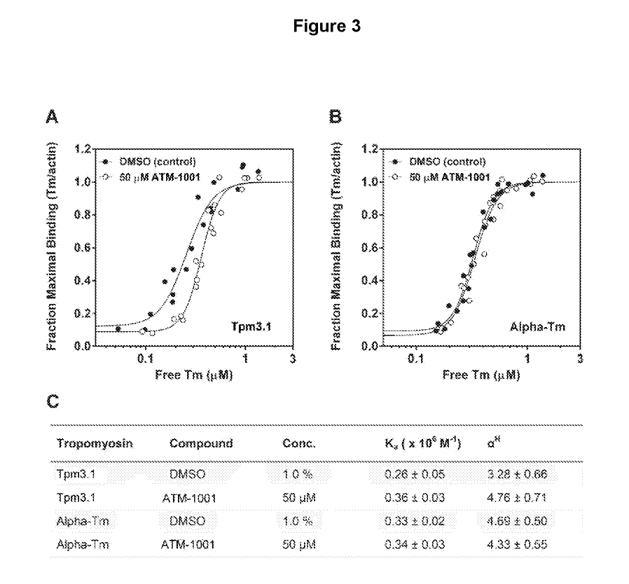

Cell free actin co-sedimentation assays were preformed to analyze the impact of compound 1001 on the polymerization of tropomyosin and subsequent association with actin. The impact of compound 1001 on Tpm3.1 binding was compared to the striated muscle isoform, alpha-fast Tm, which has an unrelated C-terminus. Compared to Tpm3.1 (FIG. 3), the binding affinity and cooperativity of alpha-fast Tm for F-actin was not affected by compound 1001 whereas compound 1001 profoundly inhibited the polymerization and association of Tpm3.1 with actin (FIG. 3A versus B). These data provide molecular evidence that compound 1001 selectively targets tumor-associated Tpm3.1 over muscle tropomyosin isoforms proving that compound 1001 elicits on-target activity.

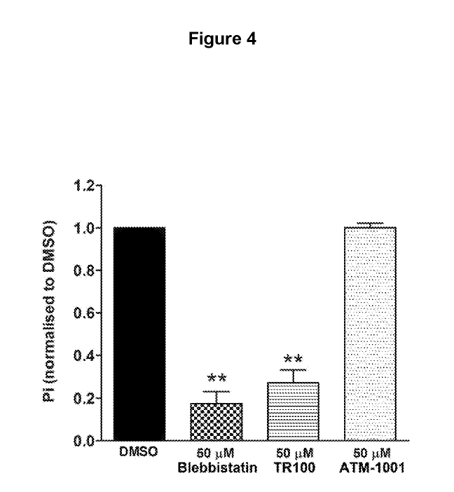

To exclude an off-target activity, the impact of compound 1001 and TR100 (another small molecule with anti-tropomyosin activity) on myosin II ATPase activity was evaluated in the absence of Tpm3.1. The small molecule blebbistatin was included as a positive control known to inhibit myosin II ATPase activity. Blebbistatin acts by binding to the myosin-ADP-Pi complex and slowing the release of inorganic phosphate (Kovacs et al., 2004). Myosin II was pre-incubated with 50 μM of blebbistatin, TR100 or 1001 prior to mixing with F-actin. As expected from previous reports, 50 μM blebbistatin strongly inhibited actin-activated myosin II ATPase activity (FIG. 4). Surprisingly, TR100 was also found to inhibit actin-activated myosin ATPase activity at levels comparable to blebbistatin (˜70%). Compound 1001 had no effect on myosin ATPase activity at equivalent concentrations to that of TR100 (FIG. 4). These data suggest that TR100 is a promiscuous small molecule, eliciting its cellular effects in a non-specific way. In contrast, compound 1001 displayed a strong on-target, dose-dependent profile in both cell viability and microfilament-disrupting assays.

Impact of Compounds of the Invention on Release of Cytokines

The ability of compounds 1004, 1007 and 1018 to inhibit the release of cytokines TNF-α, IFN-γ, IL-6, IL-21, IL-17A and IL-23 was evaluated in vitro (Tables 2 and 3). Briefly, human peripheral blood mononuclear cells (PBMCs) were isolated from human peripheral blood by Histopaque density gradient centrifugation. The freshly isolated PBMCs were seeded at 50,000 cells/well in a 96-well half area plate. PBMCs were dosed with the test compounds (at 10 μM, 1 μM and 0.1 μM) and then incubated at 37° C. and 5% CO₂ for 2 hours. To stimulate release of the cytokines IFN-γ, IL-21, IL-17A and IL-23, the PBMCs were treated with 50 ng/mL of phorbol 12-myristate 13-acetate (PMA) and 1 μg/mL of ionomycin and to stimulate the release of TNF-α and IL-6, PBMCs were treated with 100 ng/mL of lipopolysaccharide (LPS) from gram-negative bacteria. The PBMCs were then incubated at 37° C. and 5% CO₂ for a further 6 hours and the cell supernatant was collected and a Homogenous Time Resolved Fluorescence (HTRF) assay was carried out following the manufacturer's instructions. Cytokine release from the PBMCs was captured using a Perkin Elmer ENVISION 2104 microplate reader set at 615 nm and 665 nm respectively. Analysis of cytotoxicity under similar conditions using 100,000 PBMCs in a 96-well plate dosed with the same test compounds, with or without PMA and ionomycin stimulation at the 2 hour time point, revealed that any minor cell loss that had occurred, was insufficient to account for the inhibition of cytokine release observed in each of the six experiments.

TABLE 2
Inhibitory activity of compounds of the invention against a range of
cytokines in vitro.

| | % Inhibition | | | | | | | | |
|---|---|---|---|---|---|---|---|---|---|
| | TNF-α | | | IFN-γ | | | IL-6 | | |
| Compound | 10 μM | 1 μM | 0.1 μM | 10 μM | 1 μM | 0.1 μM | 10 μM | 1 μM | 0.1 μM |
| 1004 | 1 | −8 | 9 | −7 | −6 | −19 | 2 | −10 | −9 |
| 1007 | 82 | 60 | 43 | 90 | 53 | 8 | 89 | 59 | 38 |
| 1018 | 77 | 58 | 26 | 92 | 19 | 2 | 89 | 56 | 22 |

TABLE 3
Inhibitory activity of compounds of the invention against a range of
cytokines in vitro.

| | % Inhibition | | | | | | | | |
|---|---|---|---|---|---|---|---|---|---|
| | IL-21 | | | IL-17A | | | IL-23 | | |
| Compound | 10 μM | 1 μM | 0.1 μM | 10 μM | 1 μM | 0.1 μM | 10 μM | 1 μM | 0.1 μM |
| 1004 | 2 | −3 | −6 | 47 | 6 | −2 | 22 | 9 | −16 |
| 1007 | −4 | −9 | −7 | 59 | 22 | −4 | −3 | −11 | −15 |
| 1018 | 50 | 33 | 13 | 45 | 34 | 2 | 58 | −2 | −6 |

Tolerance and in Vivo Efficacy of Compound 1001

In vivo efficacy studies for compound 1001 were conducted using an A375 melanoma xenograft model in Foxn-1 nu/nu athymic mice. The human melanoma cell line A375 (sourced from American Type Culture Collection (ATCC), USA) was used for developing the flank xenograft model. Briefly, five million cells were injected subcutaneously in the right flank region of the animal. When the tumors reached 130-150 mm³ the animals were randomized into two groups, each group with 8 animals in them, so that the average tumor volume of all the groups was same.

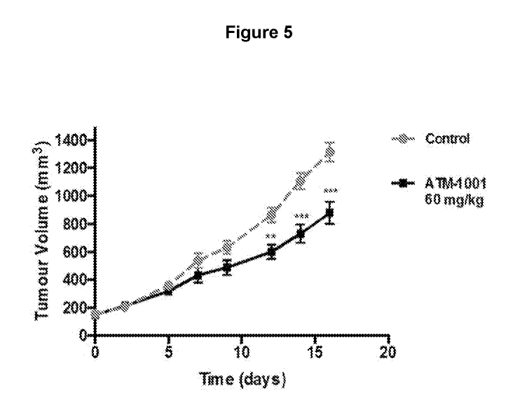

Compound 1001 was delivered intravenously (IV) at 60 mg/kg in a 30% (w/v) Captisol (cyclodextrin-containing) formulation daily for 16 days. The control group was dosed with the Captisol vehicle alone. Treatment with compound 1001 was well tolerated by the animal and resulted in a significant reduction (˜40%) in melanoma tumor growth (FIG. 5).

It will be understood that the invention disclosed and defined in this specification extends to all alternative combinations of two or more of the individual features mentioned or evident from the text or drawings. All of these different combinations constitute various alternative aspects of the invention.

### REFERENCE ARTICLES

Kovacs, M., Toth, J., Hetenyi, C., Malnasi-Csizmadia, A., and Sellers, J. R. (2004). Mechanism of blebbistatin inhibition of myosin II. J Biol Chem 279, 35557-35563.

Vindin, H., Bischof, L., Gunning, P. & Stehn, J. (2014) Validation of an algorithm to quantify changes in actin cytoskeletal organization. J Biomol Screen 19, 354-368.

1. A compound of formula (I) or a pharmaceutically acceptable drug or prodrug thereof: 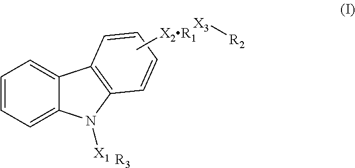
wherein:
R₁ is 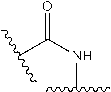 [SMILES: *NC(*)=O]
or a 5- or 6-membered carbocyclic ring wherein between 1 and 3 ring carbon atoms may optionally be replaced with S, N, O, NH or NR₅ and wherein the ring may optionally be substituted by R₆;
R₂ is a monocyclic or bicyclic carbocyclic ring having between 5 and 10 ring carbons wherein 1 or 2 ring carbon atoms may optionally be replaced with S, O, N, NH or NR₅ and wherein the ring may optionally be substituted with R₆, or R₂ is 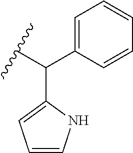 [SMILES: *C(c1ccccc1)c1ccc[nH]1]
R₃ is H, halo, NH₂, N(R₅)₂ or a 3- to 7-membered carbocyclic ring wherein between 1 and 3 ring carbon atoms may optionally be replaced by S, N, O, NH or NR₅ and wherein the ring may optionally be substituted by R₅ or R₆;
X₁ is absent, or is an alkyl group having between 1 and 10 carbon atoms, or an alkenyl group having between 2 and 10 carbon atoms;
X₂ and X₃ are independently absent or selected from the group consisting of: S, O, NH, N(R₄), CO, C(O)NH, an alkyl group having between 1 and 10 carbon atoms, an alkenyl group having between 2 and 10 carbon atoms, CH(R₄)CHC(R₄)CO, (CH₂)₀₋₅C(R₄)C(R₄)(CH₂)₀₋₅, and a 5- or 6-membered carbocyclic ring wherein between 1 and 3 ring carbon atoms may optionally be replaced by S, N, O, NH or NR₅;
X₄ is O, NH, NR₅ or S;
R₄ is H or C₁-C₆ alkyl;
R₅ is CH₃, (CH₂)₁₋₅CH₃, (CH₂)₁₋₅OMe, CF₃, CN or OCF₃; and
R₆ is H, OH, alkyl, alkenyl, halo, alkoxy, amino, alkylamino, dialkylamino or a dioxolane ring fused to 2 adjacent carbon atoms of R₁ or R₂.

2. A compound according to claim 1, wherein X₁ is absent or is an alkyl group having between 1 and 10 carbon atoms.

3. A compound according to claim 2, wherein X₁ is absent or is an alkyl group having between 1 and 5 carbon atoms.

4. A compound according to any one of the preceding claims, wherein R₃ is H, NH₂, N(R₅)₂, halo, or a 4-, 5-, 6- or 7-membered carbocyclic ring wherein between 1 and 3 ring carbon atoms may optionally be replaced by S, N, O, NH or NR₅ and wherein the ring may optionally be substituted by R₅ or R₆.

5. A compound according to claim 4, wherein the carbocyclic ring is a cycloalkyl or aryl group.

6. A compound according to any one of the preceding claims, wherein X₂ and X₃ are independently selected from the group consisting of: S, O, NH, N(R₄), C(O), C(O)NH, an alkyl group having between 1 and 10 carbon atoms, CH(R₄)CHC(R₄)C(O), and a 5-membered carbocyclic ring wherein between 1 and 3 ring carbon atoms may optionally be replaced by S, N, O, NH or NR₅.

7. A compound according to claim 6, wherein the alkyl group has between 1 and 5 carbon atoms.

8. A compound according to claim 7, wherein the alkyl group is CH₂, (CH₂)₂ or (CH₂)₃.

9. A compound according to claim 6, wherein the carbocyclic ring is an aryl group.

10. A compound according to claim 6 or 9, wherein, in the carbocyclic ring, 1 or 2 ring carbon atoms may optionally be replaced with S, N, O, NH or NR₅.

11. A compound according to claim 10, wherein, in the carbocyclic ring, the carbon atoms may optionally be replaced with N and/or O.

12. A compound according to any one of the preceding claims, wherein the compound of formula (I), or a pharmaceutically acceptable drug or prodrug thereof, is: 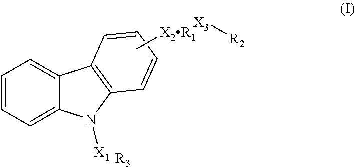
wherein:
R₁= 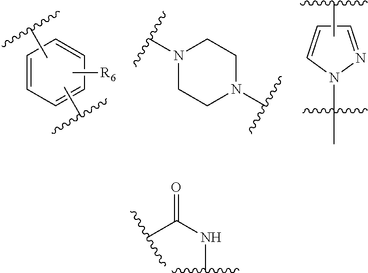
X₁=absent or (CH₂)₁₋₅
X₂ and X₃═O, NH, N(R₄), C(O), C(O)NH, (CH₂)₀₋₅, CH(R₄)CHC(R₄)C(O), pyrazole, isooxazole
R₂= 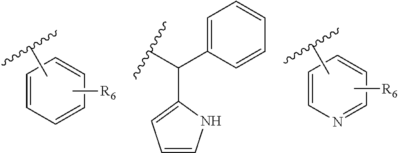
X₄═O, NH, NR₅
R₄═H, CH₃, CH₂CH₃ 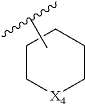
R₅═CH₃, (CH₂)₁₋₅CH₃, (CH₂)₁₋₅OMe, CF₃, CN, OCF₃
R₆═H, OH, alkyl, halo, alkoxy, amino, alkylamino, dialkyl amino, or a dioxolane ring fused to 2 adjacent carbon atoms of R₁ or R₂
R₃═H, NH₂, N(R₅)₂, halo,

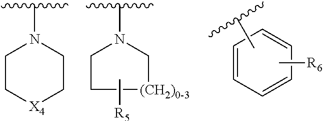

13. A compound according to any one of the preceding claims, wherein the compound is a compound of formula (Ia):

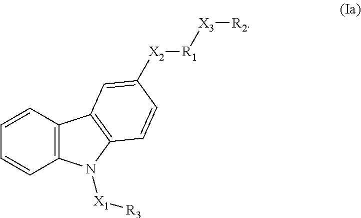

14. A compound according to any one of claims 1 to 12, wherein the compound is a compound of formula (Ib):

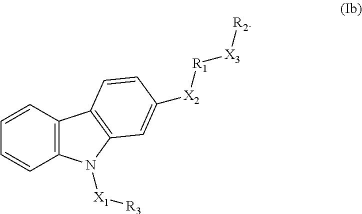

15. A compound according to any one of the preceding claims, wherein X₁ is (CH₂)₂ or (CH₂)₃.

16. A compound according to any one of the preceding claims, wherein R₃ is N(R₅)₂.

17. A compound according to claim 16, wherein R₅ is CH₃ or (CH₂)₁₋₅CH₃.

18. A compound according to claim 17, wherein R₅ is CH₂CH₃.

19. A compound according to any one of claims 1 to 15, wherein R₃ is a 4-, 5-, 6- or 7-membered cycloalkyl group wherein between 1 and 3 ring carbon atoms may optionally be replaced by S, N, O, NH or NR₅ and wherein the ring may optionally be substituted by R₅ or R₆.

20. A compound according to claim 19, wherein R₃ is:

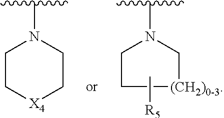

21. A compound according to claim 19, wherein R₃ is a 6-membered cycloalkyl group.

22. A compound according to claim 21, wherein R₃ is:

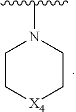

23. A compound according to claim 22, wherein X₄ is NH or NR₅.

24. A compound according to claim 23, wherein R₅ is C₁-C₆ alkyl.

25. A compound according to claim 24, wherein R₅ is CH₃ or CH₂CH₃.

26. A compound according to any one of claims 1 to 14, wherein X₁ is absent.

27. A compound according to claim 26, wherein R₃ is H.

28. A compound according to any one of claims 1 to 15, wherein R₃ is halo.

29. A compound according to claim 28, wherein R₃ is chlorine.

30. A compound according to any one of claims 1 to 15, wherein R₃ is a 5- or 6-membered aryl group wherein between 1 and 3 ring carbon atoms may optionally be replaced by S, N, O, NH or NR₅ and wherein the ring may optionally be substituted by R₅ or R₆.

31. A compound according to claim 30, wherein R₃ is:

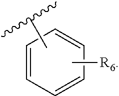

32. A compound according to claim 30 or 31, wherein R₆ is halo.

33. A compound according to claim 32, wherein R₆ is fluorine.

34. A compound according to any one of the preceding claims, wherein X₂ is C(O).

35. A compound according to any one of the preceding claims, wherein R₁ is a 5- or 6-membered cycloalkyl group wherein between 1 and 3 ring carbon atoms may optionally be replaced with S, N, O, NH or NR₅ and wherein the ring may optionally be substituted by R₆.

36. A compound according to claim 35, wherein R₁ is:

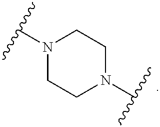 [SMILES: *N1CCN(*)CC1]

37. A compound according to any one of claims 34 to 36, wherein the compound of formula (I) is a compound of formula (Ic):

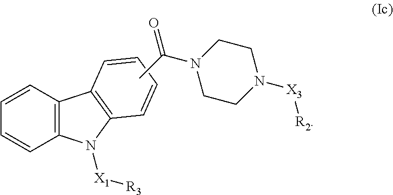

38. A compound according to any one of the preceding claims, wherein X₃ is (CH₂)₀₋₅.

39. A compound according to claim 38, wherein X₃ is CH₂, (CH₂)₂ or (CH₂)₃.

40. A compound according to any one of the preceding claims, wherein R₂ is an aryl group having 5 or 6 ring carbon atoms wherein 1 or 2 ring carbon atoms may optionally be replaced with S, O, N, NH or NR₅ and wherein the ring may optionally be substituted with R₆.

41. A compound according to claim 40, wherein R₂ is:

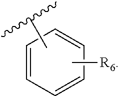

42. A compound according to claim 40 or 41, wherein R₆ is H, halo, alkoxy or a dioxolane ring fused to 2 adjacent carbon atoms of R₂.

43. A compound according to claim 42, wherein R₆ is halo.

44. A compound according to claim 43, wherein R₆ is fluorine.

45. A compound according to claim 42, wherein R₆ is alkoxy.

46. A compound according to claim 45, wherein R₆ is methoxy or ethoxy.

47. A compound according to any one of the preceding claims, wherein the compound is selected from the group consisting of:

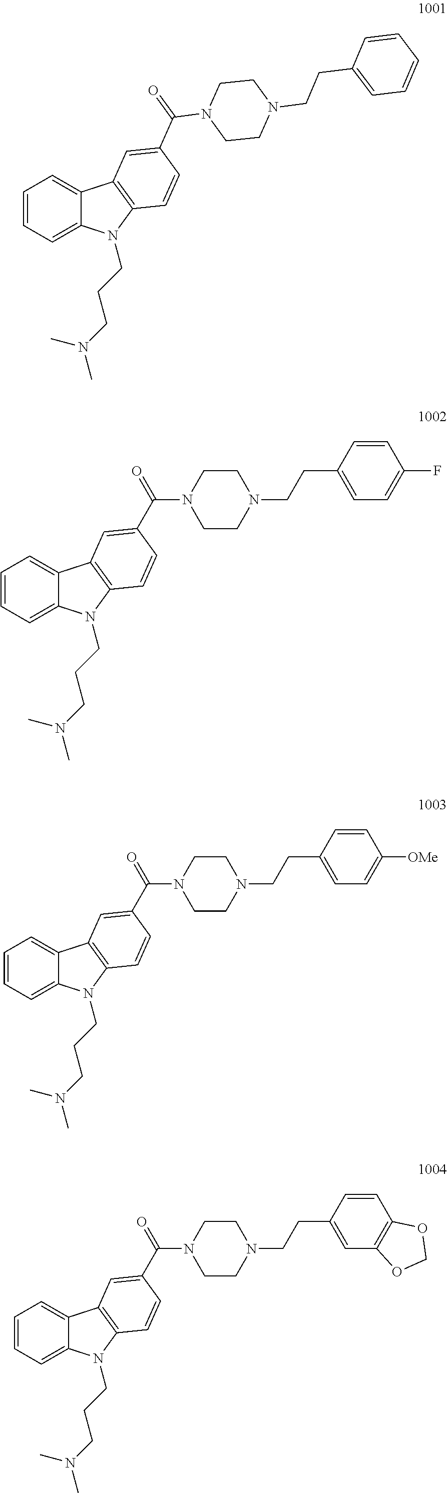 [SMILES: CN(C)CCCn1c2ccccc2c2cc(C(=O)N3CCN(CCc4ccccc4)CC3)ccc21 ; CN(C)CCCn1c2ccccc2c2cc(C(=O)N3CCN(CCc4ccc(F)cc4)CC3)ccc21 ; COc1ccc(CCN2CCN(C(=O)c3ccc4c(c3)c3ccccc3n4CCCN(C)C)CC2)cc1 ; CN(C)CCCn1c2ccccc2c2cc(C(=O)N3CCN(CCc4ccc5c(c4)OCO5)CC3)ccc21]

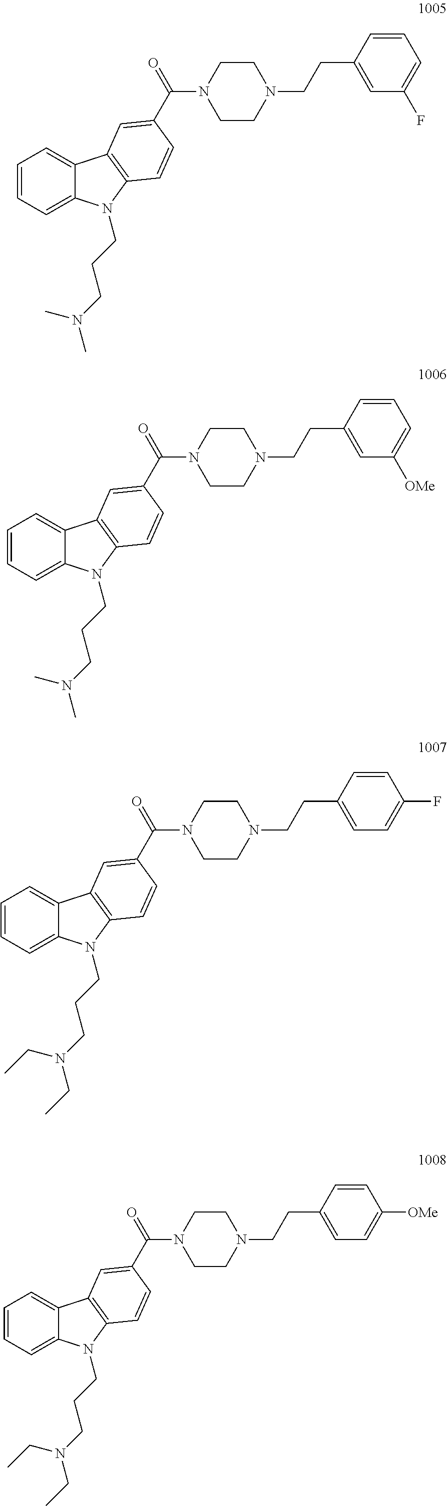 [SMILES: CN(C)CCCn1c2ccccc2c2cc(C(=O)N3CCN(CCc4cccc(F)c4)CC3)ccc21 ; COc1cccc(CCN2CCN(C(=O)c3ccc4c(c3)c3ccccc3n4CCCN(C)C)CC2)c1 ; CCN(CC)CCCn1c2ccccc2c2cc(C(=O)N3CCN(CCc4ccc(F)cc4)CC3)ccc21 ; CCN(CC)CCCn1c2ccccc2c2cc(C(=O)N3CCN(CCc4ccc(OC)cc4)CC3)ccc21]

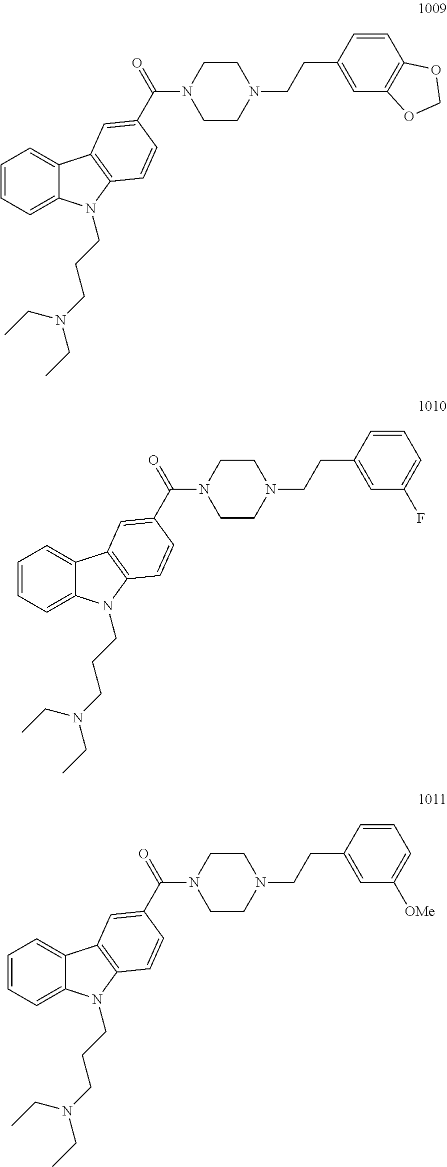 [SMILES: CCN(CC)CCCn1c2ccccc2c2cc(C(=O)N3CCN(CCc4ccc5c(c4)OCO5)CC3)ccc21 ; CCN(CC)CCCn1c2ccccc2c2cc(C(=O)N3CCN(CCc4cccc(F)c4)CC3)ccc21 ; CCN(CC)CCCn1c2ccccc2c2cc(C(=O)N3CCN(CCc4cccc(OC)c4)CC3)ccc21]

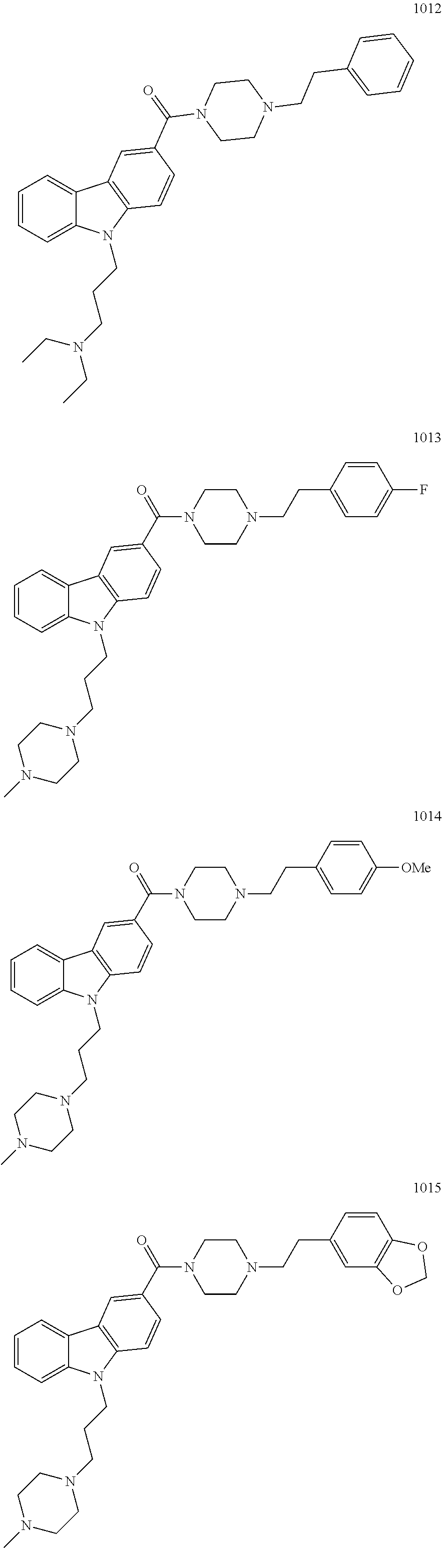 [SMILES: CCN(CC)CCCn1c2ccccc2c2cc(C(=O)N3CCN(CCc4ccccc4)CC3)ccc21 ; CN1CCN(CCCn2c3ccccc3c3cc(C(=O)N4CCN(CCc5ccc(F)cc5)CC4)ccc32)CC1 ; COc1ccc(CCN2CCN(C(=O)c3ccc4c(c3)c3ccccc3n4CCCN3CCN(C)CC3)CC2)cc1 ; CN1CCN(CCCn2c3ccccc3c3cc(C(=O)N4CCN(CCc5ccc6c(c5)OCO6)CC4)ccc32)CC1]

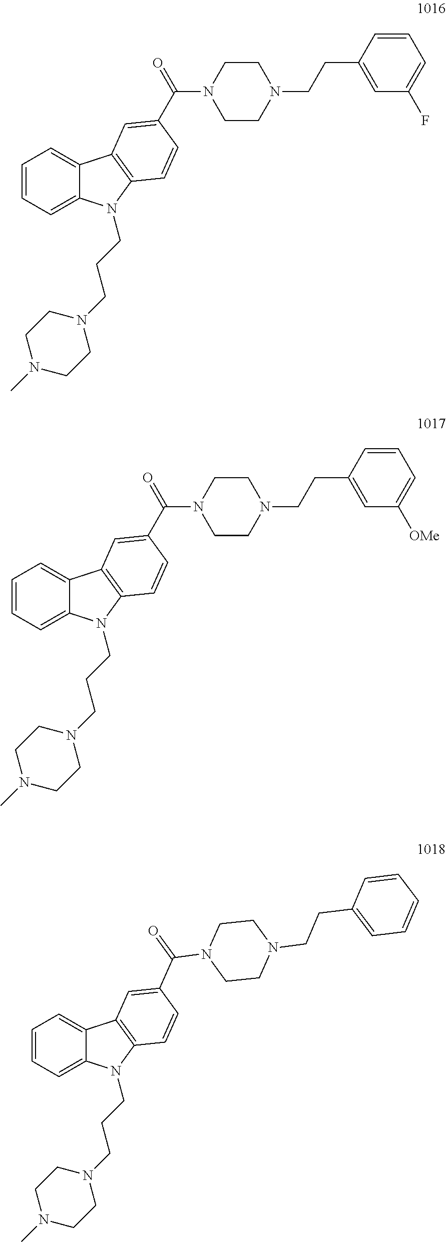 [SMILES: CN1CCN(CCCn2c3ccccc3c3cc(C(=O)N4CCN(CCc5cccc(F)c5)CC4)ccc32)CC1 ; COc1cccc(CCN2CCN(C(=O)c3ccc4c(c3)c3ccccc3n4CCCN3CCN(C)CC3)CC2)c1 ; CN1CCN(CCCn2c3ccccc3c3cc(C(=O)N4CCN(CCc5ccccc5)CC4)ccc32)CC1]

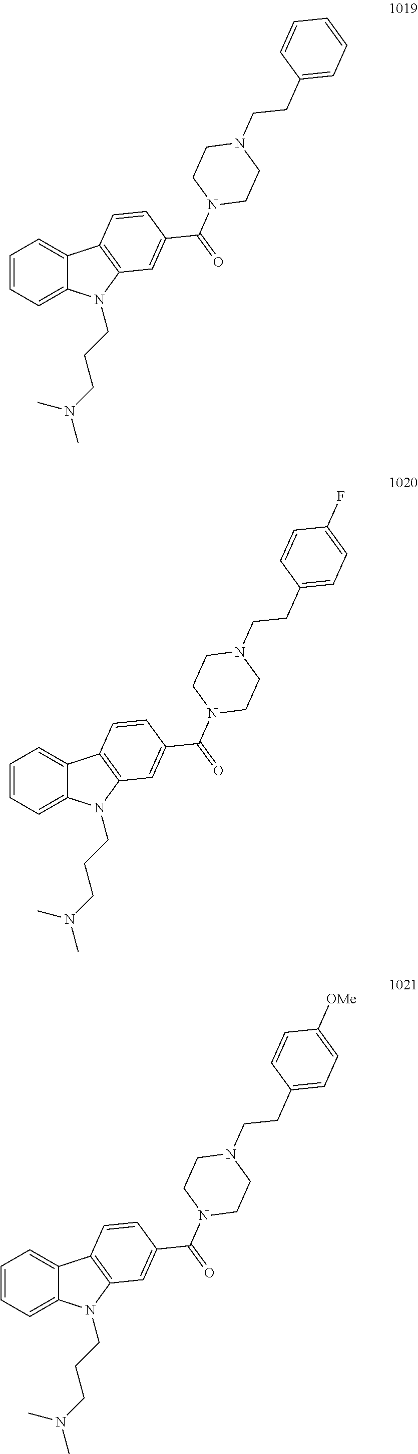 [SMILES: CN(C)CCCn1c2ccccc2c2ccc(C(=O)N3CCN(CCc4ccccc4)CC3)cc21 ; CN(C)CCCn1c2ccccc2c2ccc(C(=O)N3CCN(CCc4ccc(F)cc4)CC3)cc21 ; COc1ccc(CCN2CCN(C(=O)c3ccc4c5ccccc5n(CCCN(C)C)c4c3)CC2)cc1]

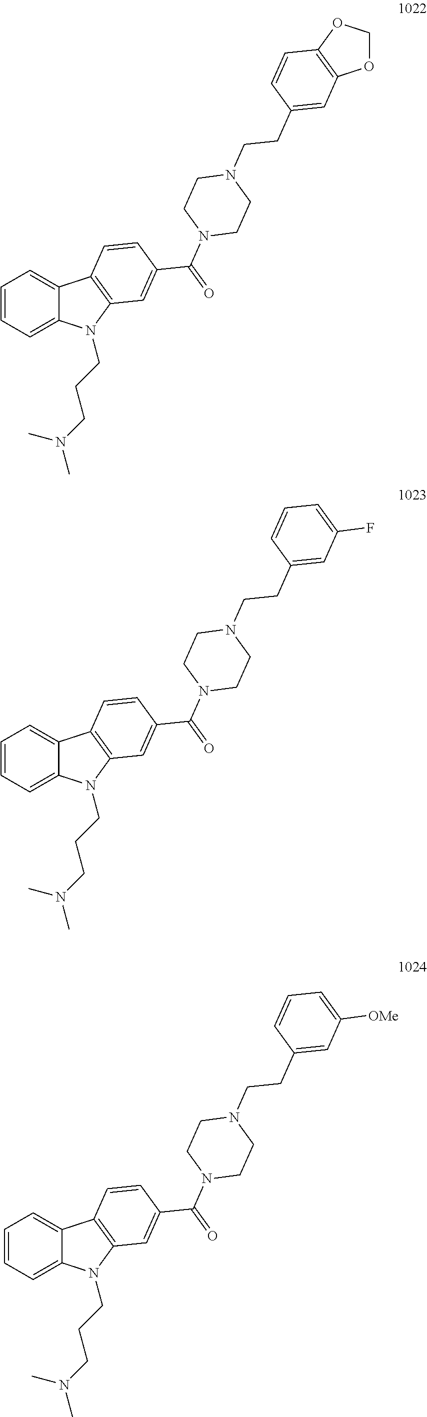 [SMILES: CN(C)CCCn1c2ccccc2c2ccc(C(=O)N3CCN(CCc4ccc5c(c4)OCO5)CC3)cc21 ; CN(C)CCCn1c2ccccc2c2ccc(C(=O)N3CCN(CCc4cccc(F)c4)CC3)cc21 ; COc1cccc(CCN2CCN(C(=O)c3ccc4c5ccccc5n(CCCN(C)C)c4c3)CC2)c1]

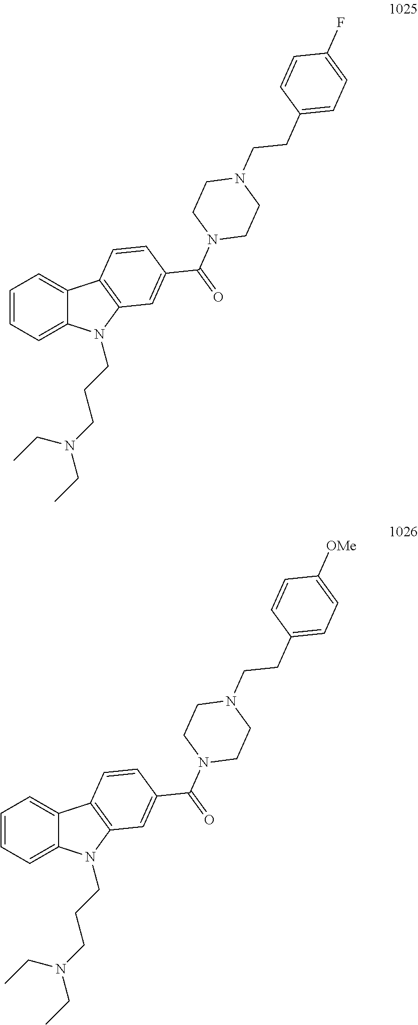 [SMILES: CCN(CC)CCCn1c2ccccc2c2ccc(C(=O)N3CCN(CCc4ccc(F)cc4)CC3)cc21 ; CCN(CC)CCCn1c2ccccc2c2ccc(C(=O)N3CCN(CCc4ccc(OC)cc4)CC3)cc21]

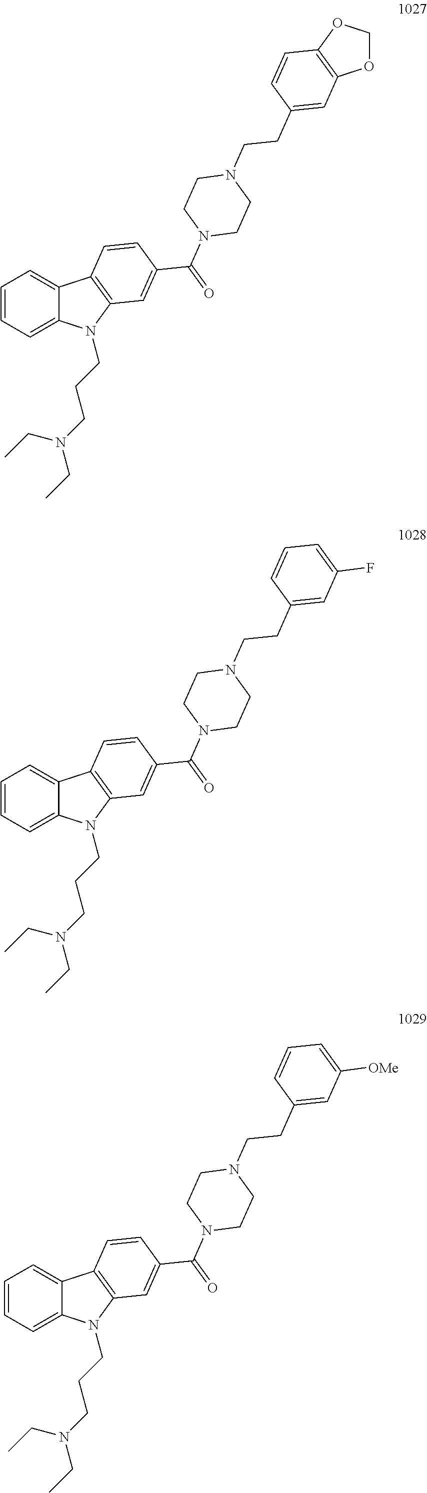 [SMILES: CCN(CC)CCCn1c2ccccc2c2ccc(C(=O)N3CCN(CCc4ccc5c(c4)OCO5)CC3)cc21 ; CCN(CC)CCCn1c2ccccc2c2ccc(C(=O)N3CCN(CCc4cccc(F)c4)CC3)cc21 ; CCN(CC)CCCn1c2ccccc2c2ccc(C(=O)N3CCN(CCc4cccc(OC)c4)CC3)cc21]

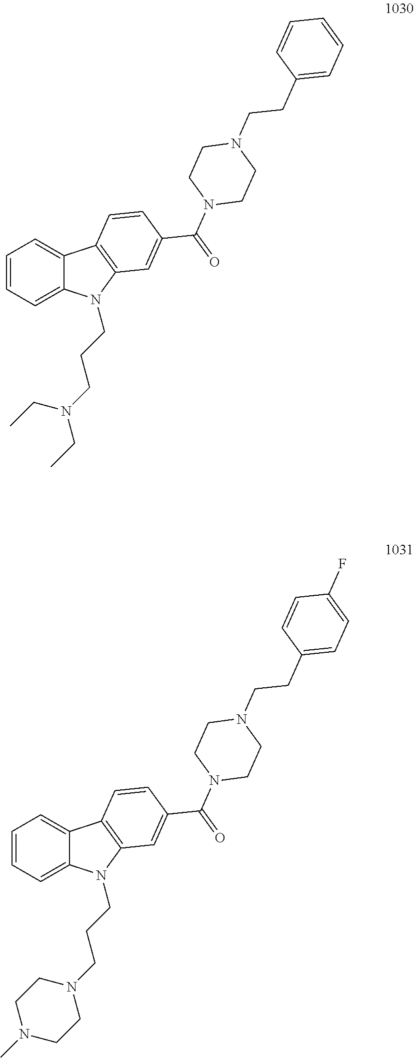 [SMILES: CCN(CC)CCCn1c2ccccc2c2ccc(C(=O)N3CCN(CCc4ccccc4)CC3)cc21 ; CN1CCN(CCCn2c3ccccc3c3ccc(C(=O)N4CCN(CCc5ccc(F)cc5)CC4)cc32)CC1]

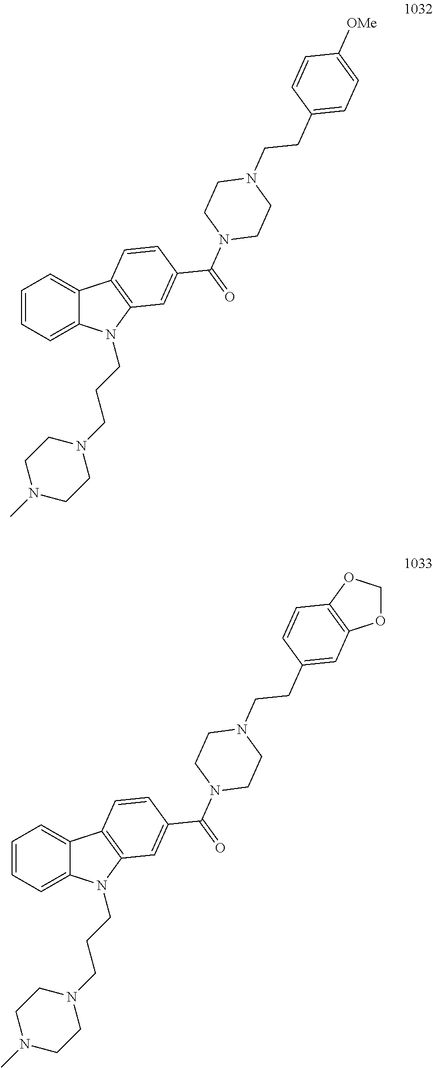 [SMILES: COc1ccc(CCN2CCN(C(=O)c3ccc4c5ccccc5n(CCCN5CCN(C)CC5)c4c3)CC2)cc1 ; CN1CCN(CCCn2c3ccccc3c3ccc(C(=O)N4CCN(CCc5ccc6c(c5)OCO6)CC4)cc32)CC1]

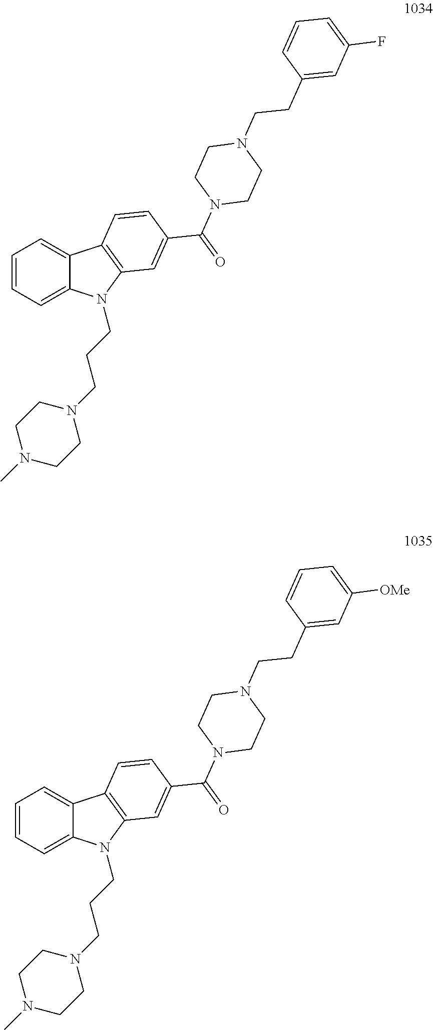 [SMILES: CN1CCN(CCCn2c3ccccc3c3ccc(C(=O)N4CCN(CCc5cccc(F)c5)CC4)cc32)CC1 ; COc1cccc(CCN2CCN(C(=O)c3ccc4c5ccccc5n(CCCN5CCN(C)CC5)c4c3)CC2)c1]

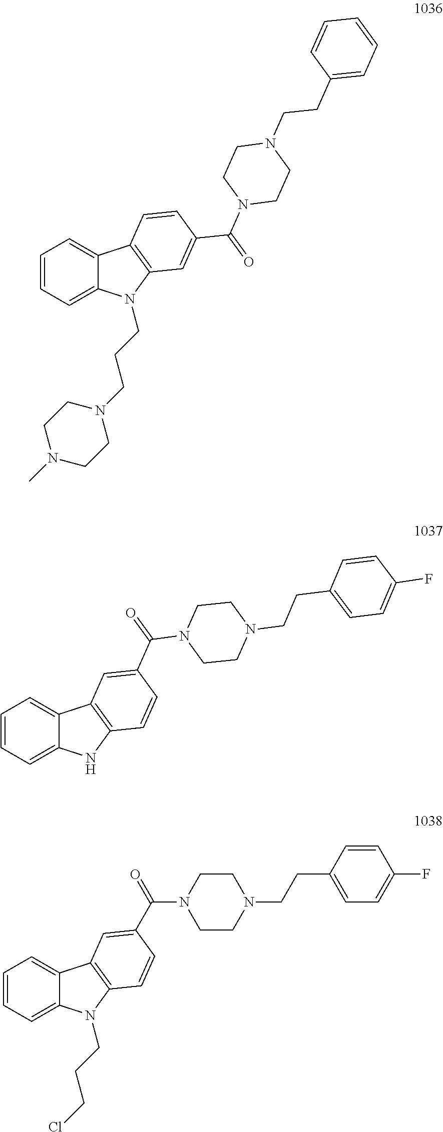 [SMILES: CN1CCN(CCCn2c3ccccc3c3ccc(C(=O)N4CCN(CCc5ccccc5)CC4)cc32)CC1 ; O=C(c1ccc2[nH]c3ccccc3c2c1)N1CCN(CCc2ccc(F)cc2)CC1 ; O=C(c1ccc2c(c1)c1ccccc1n2CCCCl)N1CCN(CCc2ccc(F)cc2)CC1]

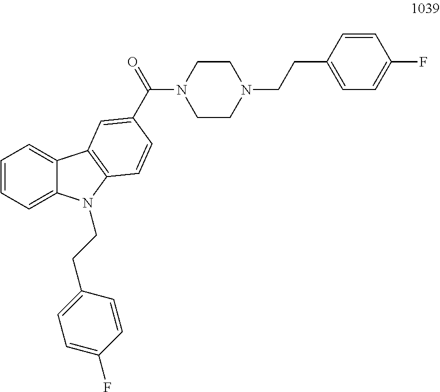 [SMILES: O=C(c1ccc2c(c1)c1ccccc1n2CCc1ccc(F)cc1)N1CCN(CCc2ccc(F)cc2)CC1]

48. A pharmaceutical composition for the treatment or prevention of a proliferative disease wherein the composition includes a compound according to any one of claims 1 to 47.

49. A method of treating or preventing a proliferative disease including administering to a subject a therapeutically effective amount of a compound according to any one of claims 1 to 47.

50. Use of a compound according to any one of claims 1 to 47 for the treatment or prevention of a proliferative disease.

51. Use of a compound according to any one of claims 1 to 47 or the pharmaceutical composition of claim 48 in the manufacture of a medicament for treating or preventing a proliferative disease.

52. A pharmaceutical composition according to claim 48, a method according to claim 49 or a use according to claim 50 or 51, wherein the proliferative disease is cancer.

53. A pharmaceutical composition for preventing the recurrence of a solid tumor wherein the composition includes a compound according to any one of claims 1 to 47.

54. A method of preventing the recurrence of a solid tumor including administering to a subject a therapeutically effective amount of a compound according to any one of claims 1 to 47.

55. Use of a compound according to any one of claims 1 to 47 for preventing the recurrence of a solid tumor.

56. Use of a compound according to any one of claims 1 to 47 or the pharmaceutical composition of claim 53 in the manufacture of a medicament for preventing the recurrence of a solid tumor.

57. A pharmaceutical composition for the treatment of an inflammatory disease or disorder wherein the composition includes a compound according to any one of claims 1 to 47.

58. A method of treating an inflammatory disease or disorder including administering to a subject a therapeutically effective amount of a compound according to any one of claims 1 to 47.

59. Use of a compound according to any one of claims 1 to 47 for treatment of an inflammatory disease or disorder.

60. Use of a compound according to any one of claims 1 to 47 or the pharmaceutical composition of claim 57 in the manufacture of a medicament for treating an inflammatory disease or disorder.

61. A pharmaceutical composition according to claim 57, a method according to claim 58 or a use according to claim 59 or 60, wherein the inflammatory disease or disorder is selected from osteoarthritis, inflammatory bowel disease, ulcerative proctitis, distal colitis, autoimmune disorders, asthma and diseases involving pulmonary inflammation, and cardiovascular disorders.
